SEQUENCE LISTING (6 sequences)

SEQ ID NO: 1 - DNA, 33 bp, synthetic construct
  source 1..33: mol_type = other DNA
tccctacacgacgctcttccgatctnnnnnnnn

SEQ ID NO: 2 - DNA, 26 bp, synthetic construct
  source 1..26: mol_type = other DNA
nnntggaattctcgggtgccaaggaa

SEQ ID NO: 3 - DNA, 70 bp, synthetic construct
  source 1..70: mol_type = other DNA
aatgatacggcgaccaccgagatctacacnnnnnnnnacactctttccctacacgacgctcttccgatct

SEQ ID NO: 4 - DNA, 65 bp, synthetic construct
  source 1..65: mol_type = other DNA
caagcagaagacggcatacgagatnnnnnnnngtgactggagttccttggcacccgagaattcca

SEQ ID NO: 5 - DNA, 28 bp, synthetic construct
  source 1..28: mol_type = other DNA
aatgatacggcgaccaccgagatctaca

SEQ ID NO: 6 - DNA, 23 bp, synthetic construct
  source 1..23: mol_type = other DNA
caagcagaagacggcatacgaga

(19) United States
(12) Patent Application Publication
(10) Pub. No.: US 2026/0085308 A1
(43) Pub. Date: Mar. 26, 2026

(54) METHODS FOR GENERATING CDNA LIBRARY FROM RNA

(71) Applicants: Jumpcode Genomics, Inc., San Diego, CA (US); The Scripps Research Institute, La Jolla, CA (US)

(72) Inventors: Keith BROWN, Carlsbad, CA (US); Steven HEAD, La Jolla, CA (US); Phillip ORDOUKHANIAN, La Jolla, CA (US)

(21) Appl. No.: 19/109,386

(22) PCT Filed: Sep. 11, 2023
(86) PCT No.: PCT/US2023/073891

§ 371 Date: Mar. 6, 2025

Related U.S. Application Data
(60) Provisional application No. 63/375,372, filed on Sep. 12, 2022.

(51) Int. Cl. C12N 15/10

(52) U.S. Cl. CPC C12N 15/1096; C12N 15/1068

(57) ABSTRACT

Disclosed herein are compositions and methods related to the fast and efficient generation of cDNA library from RNA. The method allows integration of the method in an automation adaptable system.

### CROSS-REFERENCE

This application claims priority to U.S. Provisional Application No. 63/375,372, filed on Sep. 12, 2022, which is incorporated herein by reference in its entirety.

### BACKGROUND

The disclosure herein relates to the field of molecular biology, such as methods and compositions for generating a double stranded cDNA library from RNA samples.

The processing time of a biological sample for the detection of a biomarker, pathogen, or a genetic trait in clinical samples is currently long and often requires operator attendance for running the reactions. Therefore, there is a need for developing a more viable method for nucleic acid analytical reactions which can be amenable to a partially or fully automated system and faster turnaround.

### SUMMARY OF DISCLOSURE

Provided herein are methods, compositions, and systems that constitute an improvement over preexisting methods in molecular analysis processing and archiving of a subject's DNA or RNA from biological sample in that it is fast and efficient.

Provided herein is a method of preparing a cDNA library, the method comprising: (A) obtaining a plurality of RNA molecules from a sample; (B) generating a first strand cDNA complementary to a RNA molecule from the plurality of RNA molecules using a random primer and a single stranded first adapter, (C) incorporating a ribonucleotide tail using a mixture of ribonucleotide triphosphate bases and terminal deoxynucleotidyl transferase at the 3′-terminus of the first strand cDNA, (D) adding a single stranded second adapter to the 3′-terminus of the ribonucleotide tail with a ligase whose preferred substrate is a 3′ribonucleotide termini, thereby generating a first strand cDNA with single stranded adapters at both ends; (E) amplifying the first strand cDNA, thereby preparing the cDNA library. In some embodiments, the first strand cDNA is synthesized using a random primer which is ligated to a first adapter. In some embodiments, the first adapter is single stranded. In some embodiments, the random primer is synthesized along with a first adapter.

In some aspects, the RNA is obtained from the sample, wherein the sample is a biological sample.

In some aspects, the biological sample is a fresh biological sample, a frozen biological sample or a forensic sample.

In some aspects, obtaining the plurality of RNA molecules comprises extracting total RNA from the sample.

In some aspects, the first adapter is a universal adapter.

In some aspects, the method further comprises, after generating the first strand cDNA, removing unused primers by an exonuclease and a phosphatase to degrade dNTPs, so they are not available as substrates for terminal deoxynucleotidyl transferase (TdT) in subsequent steps. In some embodiments the endonuclease is exonuclease 1 and the phosphatase is shrimp alkaline phosphatase (rSAP) Also, rSAP will remove any phosphate groups from 5′ ends of the RNA in the sample, preventing possible formation of chimeric artifact products when using T4 RNA Ligase 1 for the ligation, this is not an issue with T4 RNA ligase 2 (T4RL-2) truncated (trunc.) because it cannot use 5′phosphorylated oligos as a substrate or with T4 RNA Ligase 2, which only prefers double-stranded RNA (dsRNA or duplex RNA).

In some aspects, incorporating a ribonucleotide tail comprises incorporating less than 10 ribonucleotides at the 3′ end of the first strand cDNA by terminal deoxynucleotidyl transferase (TdT), whose preferred substrates are 3′-DNA termini and deoxy nucleotide triphosphates (dNTPs), consequently ribonucleotide triphosphates (rNTPs) are not the preferred substrate and thus only less than 10 are added to the 3′-termini of the cDNA strand.

In some aspects, the ribonucleotide tail is incorporated using a terminal deoxynucleotidyl transferase (TdT).

In some aspects, adding a single stranded second adapter comprises adding or ligating the adapter to the 3′-terminal ribonucleotide incorporated in step (C) described above.

In some aspects, amplifying comprises performing a polymerase chain reaction (PCR) using primers that anneal to the first adapter and the second adapter to generate a double stranded cDNA for the cDNA library.

In some aspects, amplified double stranded cDNA library obtained from step (E) is the first amplified cDNA library that comprises a target sequence, and unwanted non-target sequence, and wherein, the method further comprises depleting a subset of the first amplified cDNA library comprising the unwanted non-target sequence from the first amplified cDNA library.

In some aspects, depleting a subset of the first amplified cDNA library comprising the unwanted non-target sequences from the first amplified cDNA library is performed using nucleic acid guided endonuclease.

In some aspects, the nucleic acid guided endonuclease comprises CAS9 enzyme.

In some aspects, the target nucleotide sequence comprises a pathogen sequence, and the depleting a subset of the first amplified cDNA library comprises depleting non-pathogen host genomic nucleic acid.

In some aspects, depleting a subset of the first amplified cDNA library comprises depleting contaminant human genomic nucleic acid.

In some aspects, contaminant human genomic nucleic acid is ribosomal nucleic acid.

In some aspects, contaminant human genomic nucleic acid is a repeat nucleic acid sequence.

In some aspects, the target nucleotide comprises a fetal nucleic acid and the depleting a subset of the first amplified cDNA library comprises depleting non-target contaminant maternal genomic nucleic acid.

In some aspects, the target nucleotide comprises a genomic polymorphism and wherein the depleting a subset of the first amplified cDNA library comprises depleting a sequence that comprises the wild type sequence.

In one aspect, provided herein is a system for automated amplification of a target nucleotide sequence from a biological sample comprising non-target nucleotide sequences, the system comprises, (a) a control panel, operably linked to (b) programmable machine comprising a liquid handler unit with an automatic liquid suction and dispensing aspect and an optical head aspect, wherein the control panel is configured to receive input command to operate the machine.

### BRIEF DESCRIPTION OF THE DRAWINGS

Some understanding of the features and advantages of the present invention will be obtained by reference to the following detailed description that sets forth illustrative aspects, in which the principles of the invention are utilized, and the accompanying drawings.

FIG. 1 exemplifies a schematic workflow for isolation, depletion and amplification starting from nucleic acid extraction up to obtaining an enriched cDNA.

FIG. 2 depicts a schematic diagram of the workflow showing cDNA synthesis and stepwise manipulation of single stranded cDNA and resulting in amplification and generation of amplified double-stranded cDNA library (where rN is some mixture of the four possible bases of ribonucleotides; adenosine ribonucleotide, uridine ribonucleotide, guanosine ribonucleotide, and cytidine ribonucleotide that were incorporated by terminal deoxynucleotidyl transferase, TdT). The barcodes are added during the first PCR and primers are used in the second PCR next to the added barcodes from the first PCR.

FIG. 3 shows an exemplary schematic diagram of the workflow, showing details of ribonucleotide tailing by terminal deoxynucleotidyl transferase, TdT and adapter ligation where a 3′ terminal oligoribonucleotide (RNA) tail is the preferred substrate for the ligase. Ampure XP SPRI kit (commercial) is used for bead-based clean-up step. Steps 1 thru 5 occur in a successive manner without any isolation of the desired products between steps, conditions were worked out such that previous steps did not interfere with subsequent steps. There is additional possibility of using T4 RNA Ligase 1 with 5′ phosphate oligo adapter or T4 RNA Ligase 2 (duplex RNA enzyme) with a duplex RNA adapter.

FIG. 4A depicts an exemplary structure of an adenylated deoxy-oligonucleotide adapter, or App-DNA adapter sequence (with diphosphate linkage, DNA designated as black bars) to be used with T4 RNA Ligase 2 truncated (and all of its mutants, i.e., K227Q) or alternatively a 5′phosphate DNA adapter sequence to be used in conjunction with adenosine triphosphate (ATP) and T4 RNA Ligase 1. Third, an RNA duplex adapter (RNA designated as red bars) with all four ribonucleotides present in an overhang that would pair with the ribonucleotide tail of the cDNA and be ligated by T4 RNA Ligase 2 also in the presence of ATP. For all three enzymes; T4 RNA Ligase 2 truncated, T4 RNA Ligase 1, and T4 RNA Ligase 2, the 3′-RNA tail on the cDNA is the preferred substrate.

FIG. 4B shows designs of additional possible adapters that need to be used for T4 RNA Ligase 1 (a 5′phosphate oligo . . . No 5′adenylation). Also depicts the duplex adapter that would be required for T4 RNA Ligase 2.

FIG. 5A depicts a DNA gel resolution of pre-depletion cDNA libraries showing yield. The number of cycles are depicted to the right.

FIG. 5B depicts a DNA gel resolution of pre-depletion cDNA libraries for 50 ng, Ing, and 0.25 ng input RNA using either T4 RNA Ligase 2 truncated with an 5′App-deoxyoligonucleotide adapter (lanes 1-3 from left to right) or T4 RNA Ligase 1 with adenosine triphosphate (ATP) and a 5′phosphorylated deoxy oligonucleotide adapter (lanes 4-9 from left to right) in an addition reaction.

FIG. 6 depicts a DNA gel resolution of pre-depletion cDNA libraries showing yield in an exemplary preparation, PREP A. Adapter dimers are not detectable.

FIG. 7 shows comparator data of cDNA sample yields using instantly disclosed methods (A, in-house data) along with comparator data (B-C). Gel electrophoresis image of DNA resolved from cDNA library samples obtained by presently disclosed method (A) in comparison to those generated utilizing commercially available third party protocol or kit (samples B-C).

FIG. 8 shows a comparison data of post depletion ribosomal DNA sequence remaining in the samples generated using the currently disclosed method (sample A, in-house) and those generated utilizing commercially available third party protocol or kit (comparator samples B, C and D).

FIG. 9A shows % alignment and % duplicates in samples A (in-house) and C (comparator) as described above with high input RNA preparations (input concentrations indicated in the axis).

FIG. 9B shows % alignment and % duplicates in samples A (in-house), B, C and D (comparators) as described above with low input RNA preparations (input concentrations indicated in the axis).

FIG. 10A shows analysis of sequence readouts of depleted samples from preparations A and C categorized into % sequence assigned and % un-assigned (multi-mapping), un-assigned (no features) and un-assigned (ambiguity) in samples A (in-house) and C (comparator) as described above with high input RNA preparations (input concentrations indicated in the axis). The higher the assigned category, represents DNA enriched for target sequence, and removal of unwanted sequences. Category that includes un-assigned (ambiguity) may in some aspects represent non-useful sequences that are junk or disregarded.

FIG. 10B shows analysis of sequence readouts of depleted samples from different preparations, samples A, B, C and D categorized as described above with low input RNA preparations (input concentrations indicated in the axis).

FIG. 11A shows analysis of sequence readouts using star alignment program of the depleted samples from preparations A (in-house) and C (comparator) categorized into % uniquely mapped and % mapped to multiple loci, and unmapped categories as indicated, from within samples A and C as described above with high input RNA preparations (input concentrations indicated in the axis). The higher the assigned category, represents DNA enriched for target sequence, and removal of unwanted sequences. Category that includes un-assigned (ambiguity) may in some aspects represent non-useful sequences that are junk or disregarded.

FIG. 11B shows star alignment analysis of sequence readouts of depleted samples from the different preparations: samples A (in-house), B, C, and D (comparator) categorized as described above with low input RNA preparations (input concentrations indicated in the axis).

FIGS. 12A and 12B show the genes detected per million reads for each library preparation method at each input level of total RNA. The graph depicts a measure of the efficiency of the library preparation method at each of the varying total RNA input levels (A, in-house) and can even be compared across with a different library preparation method (i.e., comparator product C, B and D which select for polyadenylated RNA as described in description above).

FIG. 12A shows that at higher inputs (100 ng, 50 ng, and 10 ng) samples A (Sequencing using currently developed protocol with inhouse designed and CRISPR guides that were commercially synthesized) detects more genes per million reads than samples C which are the comparator data from a commercial source using Stranded Total RNA-Seq Kit v3—Pico Input Mammalian kit at the same inputs of total RNA.

FIG. 12B shows the comparison of the efficiency of the different library prep methods at lower total RNA input levels (1 ng, 100 pg, and 10 pg). The seq library prep with in-house designed CRISPR guides synthesized by commercial entity (samples A), at the 1 ng and 100 pg total RNA input levels, detects as many or slightly more genes per million reads than all the other methods (B, C, D).

At the 10 pg total RNA input level, the inhouse sequencing prep using CRISPR guides synthesized by a different commercial entity (samples B) for ribodepletion detects just slightly less genes per million reads as the C, or D from sequencing of the library preparations.

FIG. 13 shows data indicating ribosomal RNA alignments as percent fraction of mapped bases having ribosomal DNA sequence in a sample A preparation as described above and a commercial protocol generated sample N.

FIG. 14 shows alignment rates of reads of exemplary sequenced library preparations across the input RNA ranges for post depletion samples A and N.

FIG. 15 shows % duplicate rates of the sequenced library preparations for samples A and N post depletion.

FIG. 16 shows mean coverage of gene reads in the sequenced library preparation post depletion samples A and N at the indicated input ranges.

FIG. 17 shows the number of genes with read counts greater than 10 in the indicated samples of A and N.

FIG. 18 shows the number of genes in the sequenced library preparations with read counts greater than 10 in the indicated samples of A.

FIG. 19 shows the level of complexity with variables of the amount of RNA input using T4 RNA ligase 1. T4 RNA ligase 1 was tested with a view to replace T4 RNA ligase 2 truncated.

FIG. 20 shows an assay of a 96 sample run with control RNA, normalized input. The assay shows preliminary results in which the rRNA remaining after depletion was about 29%. Further optimization was in progress at the time the assay was completed.

FIG. 21 shows a MDS plots of the sequencing readouts of the test run using human liver RNA samples (left) and cell extracted RNA (right) library preparations from normalized input RNA.

FIG. 22A shows star alignment scores of human liver RNA library preparations in a control experiment using the protocol outlined including ribosomal RNA depletion steps, showing the number of reads, demonstrating the quality of reads following the protocol including ribosomal RNA depletion steps.

FIG. 22B shows star alignment scores of sequenced cell extracted RNA library preparations, showing the number of reads, demonstrating the quality of reads following the protocol including ribosomal RNA depletion steps.

FIG. 23A shows star alignment scores of sequences human liver RNA library preparations in a control experiment using the protocol outlined including ribosomal RNA depletion steps, showing the high sequencing read percentage, demonstrating the quality of reads following the protocol including ribosomal RNA depletion steps.

FIG. 23B shows star alignment scores of sequenced cell extracted RNA library preparations, showing the high sequencing read percentage, demonstrating the quality of reads following the protocol including ribosomal RNA depletion steps.

FIG. 24 shows the duplicate read rate, indicating another metric of the quality of reads from the sequenced library preparations.

### DETAILED DESCRIPTION

Disclosed herein are methods, systems, and compositions for a fast and reliable method of generating double stranded cDNA library from total RNA extracted from a biological sample. In some aspects, the method can be completed in a shorter amount of total amount of time (e.g., 8 hours or less) compared to the conventional methods. In some aspects, the method requires less number of reaction steps, liquid transfer steps and/or reaction vessels, and therefore increases efficiency of the process; for example, frequent changing of reaction vessels uses more time and increasing cost of production compared to conventional methods currently in use. In some aspects, the method encompasses fewer washing and elution steps. In some aspects, the method requires less number of steps requiring continuous operator presence or participation. In some aspects, for example, the advantage of the process described here includes that the operator can walk away from the reaction without disrupting the reaction process. In some aspects the reaction is amenable to full or partial automation. In some aspects, the reaction is adjustable to a bench to bedside protocol. In some aspects, the method described here has less number of active steps to completion compared to conventional methods currently used.

In one aspect, it has been observed that lack of preparedness experienced during coronavirus disease (COVID), particularly in terms of detecting the highly infectious SARS-CoV-2 pathogen, has opened the eyes of the world to a deficiency that needs to be met. It took approximately 60 days for the first reverse transcription-polymerase chain reaction (RT-PCR) tests for SARS-CoV-2 infection (developed by the United States (US) Centers for Disease Control (CDC)) to be made available. It then took more than 270 days to deploy 800,000 of these tests per day, when the estimated testing needs required over 6 million tests per day. This seriously limited ability to test and quarantine symptomatic individuals or those in close contact with confirmed positive infections, thereby allowing exponential increase in the spread of the disease. It is required that testing strategies be deployed on a population scale at ‘day zero,’ i.e., at the time of the first reported case, and for this, an increased effort in expediting every level of testing and manufacturing processes are needed. Next Generation Sequencing (NGS) might have day zero capabilities with the potential to enable feasible and broad large scale testing strategies. NGS based approaches may identify a much broader range of pathogens, resistance mutations, and biothreat pathogens than other molecular diagnostics approaches.

In one aspect, the sensitivity of SARS-CoV-2 detection via NGS can be equivalent to the sensitivity of RT-PCR detection if relevant samples are depleted of sequences likely to be irrelevant or that do not contribute to pathogen detection or host response, depending on the aim of the work. In addition, it was also shown that the such strategy may also be used for variant strain typing, co-infection detection, and individual human host response assessment, all in a single workflow using existing open-source analysis pipelines. In some aspects, the proposed NGS framework described herein may be considered pathogen agnostic, with the potential to radically transform how to pursue both large-scale pandemic response, as well as focused clinical microbiology testing in the future.

The current standard for ligation based library preparation technology requires a multitude of steps, and many washing and collection steps. Generally, it requires extraction, fragmentation, first strand cDNA synthesis, second strand cDNA synthesis, end repair, A tailing, directional ligation of adapters, CRISPR cleavage, and post cleavage amplification. Multiple bead based cleanups are also required. While these are parts of an established library construction method that allows for strand specific sequencing and unique dual indexing, it may have a number of flaws. In some aspects, a conventional system is 2 day protocol. In some aspects, a conventional system is with 78 liquid transfer steps. Second, because of the inefficiencies and the “loss” from all of the clean-up steps, minimizing such steps have led to the method described herein, that is capable of using a starting minimum sample input of 5 ng of total RNA. Third, conventional methods are not amenable to truly walk away automation starting from a sample, including extraction through library construction and depletion. In an effort to resolve this issue, the instant method is designed for multiple stepwise reactions in a vessel that require minimum intervention other than sample addition and temperature control, between washing and elution steps-which are minimized in number of times throughout the workflow. In some aspects, ideal systems would contain pre-loaded cartridges with all reagents necessary to go from a “sample” to a depleted library ready for clinical use with low manual intervention.

The method described herein provides various technical advantages that improve the process of generating nucleic acid library for sequencing (e.g., next generation sequencing), including, but not limited to lower number of processing steps than conventional methods currently used, addition-only processing steps (meant to minimize the loss of desired cDNA material that would occur in clean up steps), and/or integrating.

In some embodiments, push button cartridges may be used in order to automate the process.

In one aspect, rapid and accurate diagnosis is achieved sequencing capacity requirements must be reduced from 40 million to approximately 10 million molecules sequenced. This can be achieved by depleting additional uninformative molecules with a more comprehensive set of sgRNAs.

The following paragraphs describe a simplistic overview and an exemplary outline of the method of library preparation described herein.

In one aspect, the method comprises obtaining a biological sample. In one aspect, the biological sample comprises isolated cells, such as peripheral blood mononuclear cells (PBMCs), red blood cells, or cells from body fluids or tissues. In some aspects, the biological sample comprises excised tissues, tumor samples, peritoneal cells, bone marrow cells or other isolated cells. In some aspects, the biological sample comprises frozen tissues, or forensic tissues comprising fragmented or damaged nucleic acid material. In some aspects, the biological sample may comprise acellular nucleic acid.

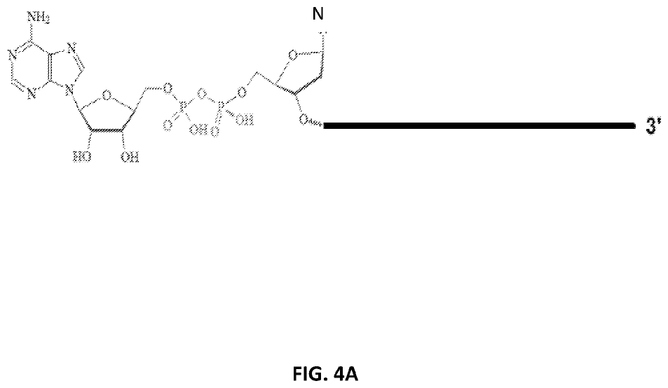

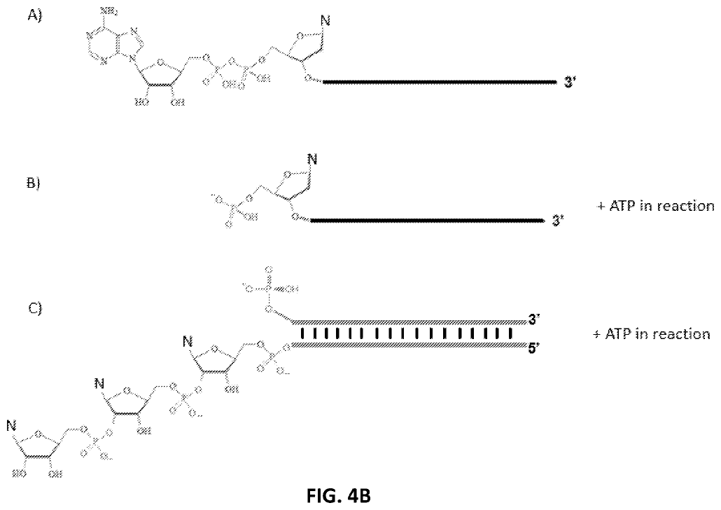

In some aspects, the method described herein may comprise the following steps. In some aspects, cell lysis and DNase digestion is performed to extract RNA from the sample. This may be followed by RNA fragmentation with heat and Mg++ for about 3 minutes. Fragmentation times may vary depending on the desired library length. In some aspects, random primers with universal adapter tails on the 5′ end are used with reverse transcriptase to create a first strand cDNA. In some aspects, exonuclease I is used to clean up excess primers and shrimp alkaline phosphatase (rSAP) is used to sequester excess dNTPs and also remove 5′phosphates from oligoribonucleotides (RNA) of the sample so they cannot be substrates for T4 RNA Ligase 1 when it is used in the addition or ligation reaction. In some aspects, a heating step at this point (approximately 95 degrees Celsius for 10 minutes) serves to 1) inactivate exonuclease I and rSAP and 2) denature the cDNA/RNA hybrid duplex, ensuring the cDNA is single-stranded, which is the preferred type of substrate for TdT as well as for T4 RNA Ligase 1, or 2 truncated. All three enzymes prefer single-stranded substrates, single-stranded DNA 3′termini for TdT and single-stranded RNA 3′termini for either ligase. T4 RNA Ligase 2 works only with duplex RNA, so would require a duplex RNA adapter to pair with the ribonucleotide tail on the cDNA. In some aspects, terminal transferase (TdT) is used to add approximately 3 ribonucleotides to the 3′ end of the cDNA strand. Truncated T4 RNA ligase (T4 RNL2 trunc) can be used to add/ligate a 5′-adenylated deoxy-oligonucleotide (5′App-DNA oligo, see FIG. 4A) adapter to the 3′ ribonucleotide tail of the cDNA molecule. Second possibility, T4 RNA Ligase 1 (with adenosine triphosphate, ATP) can also be used, instead of T4 RNL2 trunc., to ligate a 5′terminal phosphate DNA adapter (5′-p-DNA oligo, see FIG. 4B) to the 3′ ribonucleotide tail of the cDNA strand. Third possibility, T4 RNA Ligase 2 can be used with a 5′terminal phosphate double-stranded RNA adapter (5′-p-dsRNA oligo or 5′-p-duplex RNA, see FIG. 4C) 1.8× magnetic bead based cleanup (e.g., SPRI) is applied to remove excess adapter, buffer components, and isolate the desired dual adapter added cDNA products.

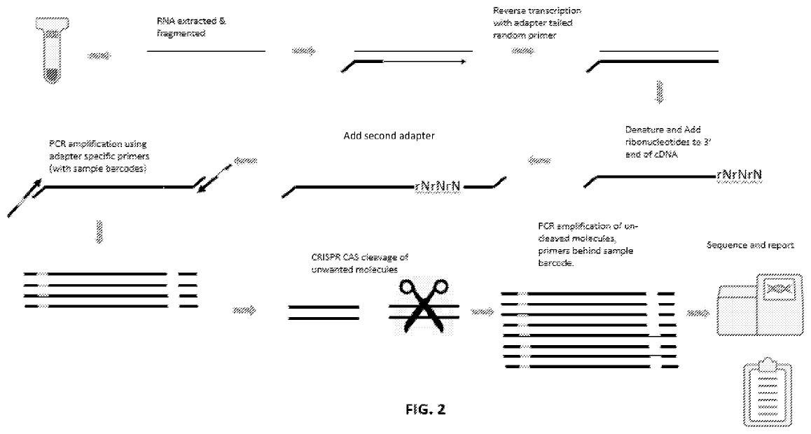

In some aspects, a first PCR is used to generate double stranded library molecules using full length PCR primers containing sample barcodes complimentary to the 3′ and 5′ adapters of the cDNA strand (see FIG. 2). Double-stranded DNA with adapters are incubated with CRISPR/CAS ribonucleoprotein complexes with guide sequences targeting unwanted molecules (list of targets for each application). 0.6×-0.8×SPRI cleanup is applied. A second post depletion PCR, with primers that target the fixed portion of the full length adapter behind the sample barcode, is applied to enrich for uncleaved library molecules of interest. The second PCR is followed by an additional SPRI cleanup. The final library is sized and quantified to enable dilution/loading on the sequencer. A DNA version would require enzymatic or physical fragmentation of the double stranded DNA, followed by denaturation before the random priming step mentioned above. The fragmentation step can be tuned to generate longer or shorter NGS library molecules with this approach for long read or short read sequencing.

In the method of cDNA library disclosed herein, in one aspect, the cDNA primer is an adapter sequence with 8 random bases at the 3′end, and in contrast to conventional methods, may not comprise a definitive/fixed sequence that is targeted to some position on the RNA itself that sequence also acts as an adapter. In some aspects the cDNA primer comprise a fixed nucleotide sequence.

In the method of cDNA library disclosed herein, in one aspect, the tailing is performed with a mixture of at least two different nucleotides of rNTPs, at least different 3 rNTPs, or all 4 different rNTPs and TdT, which contrasts to conventional methods that use just one nucleotide type.

In one aspect, it is envisioned to use the random bases added to the cDNA in conjunction with random bases (three N's) on the 5′ side of the (App-adapter) or 5′ phosphorylated adapter (p-adapter) so together will form a Unique Molecular Index (UMI) 6-8 bases in length that can be used for deduplication. This would not be possible with T4 RNA Ligase 2 and a dsRNA adapter because could not easily create the random bases in the paired portion of the duplex RNA adapter.

In one aspect, exonuclease I and shrimp alkaline phosphatase (rSAP) are added after cDNA synthesis to digest up excess adapter-N8 primer, the cDNA/RNA heteroduplex is not a substrate for exonuclease I so it is protected from digestion and rSAP is added for two purposes: first to get rid of the dNTPs from the reverse transcription reaction so they are not around for TdT tailing reaction in the subsequent step or TdT would use the dNTPs preferentially over the rNTPs, adding the DNA bases to the 3′end of the cDNA; second, to remove any biological 5′-phosphates on the total RNA sample because T4 RNA Ligase 1 could possibly form RNA-RNA chimeras between two different portions of the RNA sample, but the removal of these 5′phosphates would preclude this from occurring. In some aspects, this is followed by heating at 95° C. for 10 min (e.g., about 95° C. for about 10 minutes) to a) deactivate Exo I, and requires at least 80° C. for 20 min, b) denature cDNA/RNA, and c) because Mg2+ is present it will degrade the RNA to ˜150-200 bases allowing it to be mostly washed away in final clean up. In some aspects, addition of Na2CO3/NaHCO3 buffer may be avoided as it would require an isolation step to remove it because downstream reactions would not be able to function in it. In some aspects RNase H can be used also, but this adds additional time and enzymes to the cost and steps of the preparation, whereas heating 95° C., 10 min (e.g., about 95° C. for about 10 minutes) accomplishes the task without disrupting the flow of the preparation.

In some aspects, Cobalt is excluded from TdT tailing reaction because it has been shown to have inhibitory effect on the downstream ligation reaction. In some previously reported uses a duplex DNA adapter with dinucleotide dC's to add onto the tailed ribo G's with T4 DNA ligase, 6 hours to overnight at 16° C. reaction. This stipulation requires that the bases tailed with must be complementary to the bases in the duplex adapter since T4 DNA ligase is a duplex specific DNA ligation enzyme. A mixture of all 4 bases may not be very efficient because pairing random bases with random bases is not going to work as well as single strand ligation because of stochastic variation of the bases at each position will prevent efficient pairing. This also would be the case here with the use of T4 RNA Ligase 2 and the dsRNA adapter ligation.

The method as disclosed herein uses T4 RNA Ligase 2 truncated (or K227Q mutant or various other mutants of the enzyme available from any vendor, such as New England Biolabs) which is a single-strand specific RNA ligase, the 3′ acceptor for the addition or ligation must be an RNA terminus. In some embodiments, it may be noted that the same idea might work on DNA instead of RNA; however, the process may be highly inefficiently such that no one would use it for this application. At the same time, an addition of 5′ adenylated (App-oligo, shown in FIG. 4A) can be either DNA or RNA. In some aspects DNA is used here for the adapter sequence. In addition, in some aspects, T4 RNA Ligase 1 can also be used in conjunction with ATP as a cofactor to perform the ligation. T4 RNA Ligase 1 uses a 5′donor phosphate terminated DNA or RNA substrate (FIG. 4B, DNA used here) to ligate to the 3′ acceptor RNA termini (3′RNA is again the preferred substrate of this enzyme, 3′DNA works very inefficiently). Lastly, T4 RNA Ligase 2 works only on double-stranded RNA, requiring a duplex RNA adapter to pair with the ribonucleotide tailed cDNA molecule.

In some aspects, an 5′adenylated oligonucleotide (5′App-DNA oligo) or 5′phosphorylated (5′p-DNA oligo) or duplex RNA adapter is ligated to whatever tailed ribonucleotide bases that are on the cDNA strand (put there in the tailing reaction with TdT). Also, this reaction requires the presence of 10-20% PEG8000 to act as a crowding agent, so that the reaction will occur in 30 minutes to 1 hour (1 hour perhaps for lower inputs) at 25-28° C.

One of the advantages of the system includes that all reactions can be performed successively without isolation from reaction components, until just prior to the first PCR reaction. In some aspects steps 1, 2, 3, 4, 5 or more subsequent steps are performed in a single vessel. The final reaction mixture before PCR (containing the cDNA and all the reactants and especially the PEG8000) may be cleaned up before being added to a PCR reaction. In some aspects, if the PCR occurs in a large volume to dilute away the other components, especially the high amount of PEG). In some aspects, when used T4 DNA ligase instead of T4 RNA ligase 2 truncated with PEG8000, it may be possible to add the reaction directly to a PCR reaction that used Taq polymerase to amplify.

In the method disclosed herein, the cDNA may be cleaned up (e.g., purified) from the PEG8000 and other reactants from the series of reactions. But this cleaned up cDNA material may be added directly to a PCR reaction that uses a high-fidelity polymerase (e.g., Roche-KAPA or Watchmaker-Equinox, ˜100×Taq fidelity) instead of the lower fidelity Taq polymerase used in the publication, and both of these high fidelity enzymes are able to bypass any ribonucleotide bases that are added to the cDNA molecule.

In some aspects, the method disclosed herein can be adapted to the depletion workflow.

In some aspects, the method provides that the reactions are carried out at less time than currently used methods, kits and systems.

In some aspects, the method provides that the reactions comprise less liquid handling and transfer steps that of the currently used methods, kits and systems.

In some aspects, the method is amenable to adaptation into a hands free, mechanized robotic liquid handling system for the major fraction of the reaction.

In some aspects, the method is designed for higher efficiency and lower cost compared to the currently used methods, kits and systems.

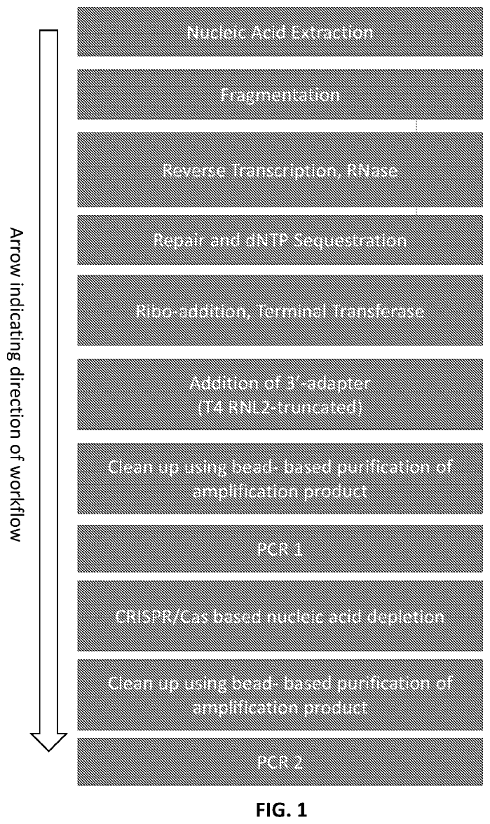

A representative time scale, broken down for each step described in the method herein is represented in FIG. 1. Some of the detailed processes are described below.

### Nucleic Acid Extraction and RNA Fragmentation:

A sample described herein is a biological sample that comprises single-copy sequence and multi-copy sequence. A biological sample may be any sample derived from an organism that may comprise cells tissues, or subcellular materials, including nucleic acids. In some cases a sample is fragmented and differentially degraded. In some aspects, a sample or a biological sample comprises blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. In some cases, a blood sample comprises circulating tumor cells or cell free nucleic acids, for example, tumor RNA, fetal RNA or cell free RNA. In some embodiments, RNA is extracted from tissue, cell or biological sample provided.

Provided herein are methods, compositions and kits related to the selective enrichment of nucleic acid extraction and modifications that further allow downstream functions, such as selective enrichment of pathogen nucleic acids, symbiote nucleic acids, microbiome nucleic acids, high information regions, cancer alleles, or other nucleic acids of interest in a sample.

In some cases, the sample comprises a combination of a host sample such as a human, cow, horse, sheep, pig, monkey, dog, cat, gerbil, bird, mouse, and rat, or any mammalian laboratory model for a disease, condition or other phenomenon involving rare nucleic acids. In some cases, the host nucleic acid is from a human. A host may be considered as an organism that harbors a parasite, a pathogen or a benign or a relatively benign microorganism. In some aspects, the sample may be contaminated with a second nucleic acid sample. Some of examples of the second nucleic acid, e.g., the nucleic acid of interest can be from pathogens, microbiomes, tumor, fetal RNA in a maternal sample, alleles, and mutant alleles. In some cases, the second nucleic acid is from a non-host. In some cases, the second nucleic acid is from a prokaryotic organism. In some cases, the second nucleic acid is from one or more selected from the group consisting of a eukaryote, virus, bacterial, fungus, and protozoa. In some aspects, the second nucleic acid can be from tumor cells. In some aspects, the second nucleic acid can be fetal RNA in a maternal sample. In some aspects, the second nucleic acid can be alleles or mutant alleles. Microbiomes are also sources of second nucleic acids consistent with the disclosure herein, as are other examples apparent to one of skill in the art.

In some aspects, one or more RNA molecules present in the sample is a synthetically prepared wherein the RNA may comprise a 2′ modified nucleoside, such as a 2′-O-modified ribose, a 2′-O-methyl nucleoside, or a 2′-O-methoxyethyl nucleoside.

Total RNA extraction and RNA fragmentation are performed according to known methods. In some aspects, the fragmentation is performed using a Hybrid A N8 primer (commercially available or synthesized). In some aspects, the Hybrid A N8 primer is HPLC purified. In some aspects, the fragmentation yields fragments from the total extracted RNA is 10-10000 bases long. In some aspects, the fragments are 10 bp to about 1000 bp. In some cases, the second nucleic acid capped with an adapter having a size in a range from about 10 bp to about 1000 bp. In some aspects, the fragments are from about 25 bp to about 2000 bp. In some aspects, the fragments are from about 25 bp to about 2000 bp. In some aspects, the fragments are from about 50 bp to about 5000 bp. In some aspects, the fragments are about 100 bp to about 10000 bp.

Preparing Single Stranded eDNA with 5′ and 3′ Adapters and UMI.

In some aspects, provided herein is a method for preparing cDNA with increased efficiency for generating a cDNA library. In one aspect, the first strand cDNA generated by reverse transcriptase is ligated with two single stranded adapters at either side, i.e., at the 5′ and 3′ ends. In some embodiments, one of the two single stranded adapters may be a universal adapter. in some embodiments, one of the two single stranded adapters may comprises a sequence from strand of a double stranded universal adapter. In some embodiments at least one of the two single stranded adapters comprises a unique sequence. In some aspects, the adapter ligated single stranded cDNA comprises unique molecular identifiers (UMIs). In some embodiments, the described method is a fast and easy method of adding/ligating single stranded adapters to a single stranded cDNA molecule, wherein the adapters are distinct (e.g., wherein the 5′ adapter is distinct from the 3′ adapter). In some aspects, the 3′ adapters can have a sequence that is unique. In some aspects, the 3 adapter can have 1, 2, 3, 4 or more unique nucleotides added at the terminal prior to ligation to the single stranded cDNA molecule. In some aspects, the method does not include duplex DNA ligation reactions. In some aspects, the method does not include T4 DNA ligation enzyme reactions. As such, in such aspects, the method does not comprise at least one or more time consuming reaction processes to so reduce the overall reaction time and increase the efficiency. In some aspects, the excess unused primers are removed from the reaction by adding exonuclease I and shrimp alkaline phosphatase (rSAP) to remove dNTPs and eliminate possible chimera artifacts from the use of T4 RNA Ligase I

First Strand cDNA Synthesis: In some aspects, a first strand cDNA synthesis is prepared from total RNA extracted from a biological sample. In some aspects, a first strand synthesis is performed using a polyA+RNA, using oligo dT primers. In some aspects, for generating a cDNA that can comprises a 5′ adapter and a 3′ adapter, as is disclosed herein, a set of oligonucleotide random primers are generated, wherein a 5′ single stranded adapter is attached to random primers and used for reverse transcription, e.g., the 5′ adapter sequence terminates in a random primer sequence at the 3′ end. Likewise, the method can be adapted to generating oligo dT primers having the 5′ adapter sequence. In some aspects, the fragmented total RNA, or alternatively the purified poly A+RNA is subjected to reverse transcription reaction using a suitable reverse transcriptase and primers wherein the primers comprise the 5′-adapter sequence. In some aspects, the 5′ adapter is 3, 4, 5, 6, 7, 8, 9, 10, 11, 12, 13, 14, 15 or more deoxyribonucleotides long. In some aspects the 5′ adapter comprises a barcode sequence. In some aspects, the barcode is a 2-5 nucleotide sequence. In some aspects, a random primer is 5, 6, 7, 8, 9, 10, 11, 12, 13, 14, 15, 16, 17, 18, 19, 20, 21 or more deoxyribonucleotides long. In some aspects. A high fidelity reverse transcriptase is used for the reaction. An example of high fidelity reverse transcription is MMLV RT. In some aspects the amount of starting material can be 1-100 pg RNA, 1-1000 pg RNA, 1 pg-10 mg RNA, 1 pg-100 mg RNA, 1 pg-500 mg RNA, 1 pg-1 mg RNA. RNA targets can be from 100 bp to 20 kb in length. The temperature at which first strand synthesis reaction is performed can vary as per the recommendations of the manufacturer of the enzyme. Usually, a temperature range of 42-55° C. is recommended for most high fidelity PCT reactions.

Ribo-tailing First Strand cDNA: In some aspects, the 3′ end of the cDNA strand is modified by the addition of between two and four ribonucleotides. In some aspects, incorporating a short ribonucleotide tail comprises incorporating 2, 3, 4 or more ribonucleotides at the 3′ end of the first strand cDNA. In some aspects, the ribonucleotides incorporated at the 3′ end are not repeat oligonucleotides. In some aspects, the ribonucleotides incorporated at the 3′ end are not all Guanidine ribonucleotides. However, it may be understood that a mixture of all 4 bases would not be very efficient because pairing random bases with random bases would reduce the yield and efficiency. In some aspects, terminal transferase (TdT) is used for incorporating the 2, 3, 4 or more ribonucleotides. In some aspects, a single strand specific RNA ligase is used for incorporating the 2, 3, 4 or more ribonucleotides to the 3′ end of the first strand cDNA. In some aspects, cobalt is excluded from TdT tailing reaction. In some aspects, inclusion of cobalt can affect or minimize the efficiency of downstream ligation reaction.

Adapter addition: In some aspects, adding a single stranded second adapter comprises adding the adapter to the 3′-terminal ribonucleotide incorporated. In some aspects the addition is performed by a single strand RNA ligase, for which the 3′ acceptor for the addition is an ribonucleotide. In some aspects, the ligase is T4 RNA Ligase 2 truncated (or KQ mutant from New England Biolabs, or any other mutants). In some aspects, the terminal nucleotide is an adenylate base. In some aspects, the adenylate (A) base is a deoxyribonucleotide or a ribonucleotide. In one aspect, in order to increase the efficiency of the ligase reaction, the 5′-oligonucleotide at the addition/ligation end is designed to be an adenine (A) base. In some aspects, the adenylate base is a deoxyribonucleotide. In some aspects, the adenylate base is a ribonucleotide. In some aspects, the adapter comprises an adenylate base. In some aspects, the adenylate base is incorporated at the 5′ terminus of the adapter. In some aspects, the 5′ adenylation is a modification of a DNA oligonucleotide adapter. In some aspects, used herein is a pre-adenylated, designed for generating 5′ pre-adenylated DNA/RNA oligonucleotides. In some aspects, adenylated oligos with a pyrophosphate linkage are substrates of T4 RNA ligase in the absence of ATP, which can significantly reduce undesired self-ligation and other side products. Preferably the adapter sequence is 10-30 nucleotides long. The adapter sequence may be 10-25 nucleotides in length. The adapter sequence may be 10-20 nucleotides in length. The adapter sequence may be 15-30 nucleotides in length. The adapter sequence may be 20-30 nucleotides in length. The adapter sequence may be 15-25 nucleotides in length. The adapter sequence may be 15-20 nucleotides in length. The adapter sequence may be 20-25 nucleotides in length. The adapter sequence may be 22-25 nucleotides in length. The adapter sequence may be 15 nucleotides in length. The adapter sequence may be 16 nucleotides in length. The adapter sequence may be 17 nucleotides in length. The adapter sequence may be 18 nucleotides in length. The adapter sequence may be 19 nucleotides in length. The adapter sequence may be 20 nucleotides in length. The adapter sequence may be 21 nucleotides in length. The adapter sequence may be 22 nucleotides in length. The adapter sequence may be 23 nucleotides in length. The adapter sequence may be 24 nucleotides in length. The adapter sequence may be 25 nucleotides in length.

In some aspects, the adapter is App-B adapter. An exemplary App-B adapter may have a nucleotide sequence represented as 5′-App-NNN-adapter-3′. In some aspects, the adenylated App-RNA oligonucleotide RNA is added or ligated to the ribotailed cDNA using the T4 RNA ligase 2 truncated as described above. In some aspects, adenylated App-DNA oligonucleotide is added to ribotailed cDNA strand using the T4 RNA Ligase 2 truncated described above. In some aspects the adenylate base used in the reaction is as shown in FIG. 4. In some aspects, the reaction mixture comprises the TdT reaction components, in which the adenylated App-DNA oligonucleotide ligase reaction reagents are added at suitable time of incubation and at suitable temperatures for each reaction to occur, as prescribed by the manufacturers of the respective enzymes. In some aspects, the reaction is performed at optimized temperatures and conditions for the reactions. In some aspects, the reaction is performed in presence of PEG8000 to act as a crowding agent. In some aspects, about 1%, 2%, 3%, 4%, 5%, 6%, 7%, 8%, 9%, 10%, 11%, 12%, 13%, 14%, 15%, 16%, 17%, 18%, 19%, 20% PEG8000 is used. In some aspects, about 10%-20% PEG is used. In some aspects, about 15% PEG8000 is used. In some aspects, care is taken for effective mixing the reaction components because the reaction mixture can be viscous.

In some aspects, the above step is followed by a clean-up reaction.

In some aspects, a clean-up reaction is carried out in order to remove spent or unwanted reactants or byproducts from nucleic acid product obtained at the end of a reaction. In one aspect, the nucleic acid product is a DNA. In some aspects, the nucleic acid product is an RNA. In some aspects, the DNA is PCR amplified DNA. In some aspects, a clean-up reaction may be undertaken following a PCR reaction, a reverse transcriptase reaction, an end-labeling reaction, a genomic DNA or RNA extraction, a subtractive hybridization or any such applications, with the purpose to remove from the product dNTPs, spent or excess enzymes, salts and other reagents, fragments of DNA that are less than optimal than the size of the nucleic acid product, in order to obtain a clean PCR product. In some aspects, a clean-up reaction is carried out to remove reagents, reactants or byproducts that are likely to interfere with subsequent downstream reactions, for example sequencing, restriction digestion, labeling, addition, cloning, in vitro transcription, blotting or in situ hybridization. In some aspects, a clean-up reaction is carried out using a commercial kit. In some aspects, a clean-up reaction can be performed without a commercially available kit. In some aspects, a clean-up reaction may involve binding of the desired nucleic acid product to a sold phase, e.g., a membrane, washing to remove unwanted spent or excess reagents, reactants or byproducts, and followed by elution of the desired nucleic acid.

In some aspects, amplifying comprises performing a polymerase chain reaction (PCR) using primers that anneal to the first adapter and the second adapter to generate a double stranded cDNA. In some aspects, amplification of the double stranded cDNA is performed by PCR to generate a double stranded cDNA library. In the method described herein, the cDNA must be cleaned up from the PEG8000 and other reactants from the series of reactions. But this cleaned up cDNA material is added directly to a PCR reaction that uses a high-fidelity polymerase (Roche-KAPA or Watchmaker-Equinox, ˜100×Taq fidelity) instead of the lower fidelity Taq polymerase used in the publication, and both of these high fidelity enzymes are able to bypass any ribose bases that are added to the cDNA molecule.

In some aspects, an amplification is continued for 5 cycles, 8 cycles, 10 cycles, 15 cycles, 20 cycles, 25 cycles, 30 cycles, 35 cycles, 40 cycles, 45 cycles, 50 cycles, 55 cycles or 60 cycles. A cycle, as is known to one of skill in the art are thermal cycles that allowing in a cyclic manner the functions: primer annealing, polymerase reaction, and denaturation, followed by primer annealing during the amplification reactions such that with each cycle, DNA is amplified in an exponential phase.

In some aspects, the amount of DNA for starting an amplification reaction is as low as 5 μg of DNA. In some aspects, the amount of DNA for starting an amplification reaction is as low as 10 pg. In some aspects, the amount of DNA for starting an amplification reaction is 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 pg. In some aspects, the amount of DNA for starting an amplification reaction is as low as 0.2 ng DNA. In some aspects, the amount of DNA for starting an amplification reaction is as low as 0.3, 0.4, 0.5, 0.6, 0.7, 0.8, 0.9, or 1 ng DNA. In some aspects, the amount of DNA for starting an amplification reaction is as low as 2 ng DNA, 5 ng DNA or 10 ng DNA. In some aspects, the amount of DNA for starting an amplification reaction is 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890 900, 910, 920, 930, 940, 950, 960, 970, 980, 990, 1000 ng.

The method described herein to generate a double stranded DNA can comprise sequences amplified total RNA, a large fraction of which may not comprise a target sequence desired to be amplified for diagnostic purpose or library preparation for future use. In some aspects, the PCR amplified DNA this obtained is subjected to depletion of unwanted sequences using selective removal of the unwanted sequences. In some aspects, the method encompasses enrichment of selective sequences by removal of unwanted sequences.

### Depletion Workflow

Endonuclease for targeted cleavage of nucleic acid: Methods disclosed herein comprise targeting cleavage of the first nucleic acid using a site-specific, targetable, and/or engineered nuclease or nuclease system. In one embodiment, a target may be considered as a specific sequence on a nucleic acid that is required to be cleaved. In some embodiments, a target may refer to the type or the source of the nucleic acid that is desired to be cleaved. A nuclease may be considered targetable when the nuclease may be designed to act only at the specific target, e.g. cleave the target. Such nucleases may create double-stranded break (DSBs) at desired locations in a genomic, cDNA or other nucleic acid molecule. In other examples, a nuclease may create a single strand break. In some cases, two nucleases are used, each of which generates a single strand break. Many cleavage enzymes consistent with the disclosure herein share a trait that they yield molecules having an end accessible for single stranded or double stranded exonuclease activity.

The endonuclease used herein can be a restriction enzyme specific to at least one site on the first nucleic acid and that does not cleave a second nucleic acid. The endonuclease described herein can be specific to a repetitive nucleic sequence in a host genome, such as a transposon or other repeat, a centromeric region, or other repeat sequence. For example, some restriction endonucleases consistent with the disclosure herein are Alu specific restriction enzymes. A restriction is Alu specific or, for that matter, other target ‘specific’ if it cuts a target and does not cut other substrates, or cuts other targets infrequently so as to differentially deplete its ‘specific’ target. The presence of a non-Alu or other non-target cleavage, such as due to the rare occurrence of the cleavage site elsewhere in a host genome or transcriptome, or in a pathogen or other rare nucleic acid present in a sample, does not render an endonuclease ‘nonspecific’ so long as differential depletion of undesired nucleic acid is effected.

The first nucleic acid can include a restriction enzyme Alu recognition site. The second nucleic acid does not include the Alu recognition site. In some aspects, the first nucleic acid comprises at least one sequence that maps to at least one nucleic acid recognition site selected from the group consisting of recognition sites of AluI, AsuHPI, Bpu10I, BssECI, BstDEI, BstMAI, HinfI, and BstTUI. In some aspects, the second nucleic acid does not include at least one of the recognition sites selected from recognition sites of AluI, AsuHPI, Bpu10I, BssECI, BstDEI, BstMAI, HinfI, and BstTUI.

Endonucleases consistent with the disclosure herein variously include at least one selected from Clustered Regulatory Interspaced Short Palindromic Repeat (CRISPR)/Cas system protein-gRNA complexes, Zinc Finger Nucleases (ZFN), and Transcription activator like effector nucleases. In some aspects, the gRNAs, or in other words, the guide RNAs, are complementary to at least one site on the first nucleic acid to generate cleaved first nucleic acids capped only on one end. Other programmable, nucleic acid sequence specific endonucleases are also consistent with the disclosure herein. Such endonucleases may be targetable, and may be further engineered to act on the specific target.

Engineered nucleases such as zinc finger nucleases (ZFNs), Transcription Activator-Like Effector Nucleases (TALENs), engineered homing endonucleases, and RNA or DNA guided endonucleases, such as CRISPR/Cas such as Cas9 or CPF1, and/or Argonaute systems, are particularly appropriate to carry out some of the methods of the present disclosure. Additionally or alternatively, RNA targeting systems may be used, such as CRISPR/Cas systems including c2c2 nucleases.

Methods disclosed herein may comprise cleaving a target nucleic acid using CRISPR systems, such as a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. Alternatively, Class 2 systems include a single effector molecule and include Type II. Type V, and Type VI.

CRISPR systems used in some methods disclosed herein may comprise a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence. CRISPR systems may comprise a single or multiple guiding RNAs. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs. In some cases, target nucleic acids do not comprise a PAM or PFS.

A gRNA may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some examples, the spacer sequence may be less than 10 or more than 36 nucleotides in length.

A gRNA may comprise a repeat sequence. In some cases, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the spacer sequence may be less than 10 or more than 50 nucleotides in length.

A gRNA may comprise one or more synthetic nucleotides, non-naturally occurring nucleotides, nucleotides with a modification, deoxyribonucleotide, or any combination thereof. Additionally or alternatively, a gRNA may comprise a hairpin, linker region, single stranded region, double stranded region, or any combination thereof. Additionally or alternatively, a gRNA may comprise a signaling or reporter molecule.

A CRISPR nuclease may be endogenously or recombinantly expressed. A CRISPR nuclease may be encoded on a chromosome, extrachromosomally, or on a plasmid, synthetic chromosome, or artificial chromosome. A CRISPR nuclease may be provided as a polypeptide or mRNA encoding the polypeptide. In such examples, polypeptide or mRNA may be delivered through standard mechanisms known in the art, such as through the use of cell permeable peptides, nanoparticles, or viral particles.

gRNAs may be encoded by genetic or episomal DNA. gRNAs may be provided or delivered concomitantly with a CRISPR nuclease or sequentially. Guide RNAs may be chemically synthesized, in vitro transcribed or otherwise generated using standard RNA generation techniques known in the art.

A CRISPR system may be a Type II CRISPR system, for example a Cas9 system. The Type II nuclease may comprise a single effector protein, which, in some cases, comprises a RuvC and HNH nuclease domains. In some cases a functional Type II nuclease may comprise two or more polypeptides, each of which comprises a nuclease domain or fragment thereof. The target nucleic acid sequences may comprise a 3′ protospacer adjacent motif (PAM). In some examples, the PAM may be 5′ of the target nucleic acid. Guide RNAs (gRNA) may comprise a single chimeric gRNA, which contains both crRNA and tracrRNA sequences. Alternatively, the gRNA may comprise a set of two RNAs, for example a crRNA and a tracrRNA. The Type II nuclease may generate a double strand break, which is some cases creates two blunt ends. In some cases, the Type II CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type II nickase is used to generate two single strand breaks, the resulting nucleic acid free ends may either be blunt, have a 3′ overhang, or a 5′ overhang. In some examples, a Type II nuclease may be catalytically dead such that it binds to a target sequence, but does not cleave. For example, a Type II nuclease may have mutations in both the RuvC and HNH domains, thereby rendering both nuclease domains non-functional. A Type II CRISPR system may be one of three sub-types, namely Type II-A, Type II-B, or Type II-C.

A CRISPR system may be a Type V CRISPR system, for example a Cpf1, C2c1, or C2c3 system. The Type V nuclease may comprise a single effector protein, which in some cases comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some cases, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which in some cases generates a 5′ overhang. In some cases, the Type V CRISPR nuclease is engineered to be a nickase such that the nuclease only generates a single strand break. In such cases, two distinct nucleic acid sequences may be targeted by gRNAs such that two single strand breaks are generated by the nickase. In some examples, the two single strand breaks effectively create a double strand break. In some cases where a Type V nickase is used to generate two single strand breaks, the resulting nucleic acid free ends may either be blunt, have a 3′ overhang, or a 5′ overhang. In some examples, a Type V nuclease may be catalytically dead such that it binds to a target sequence, but does not cleave. For example, a Type V nuclease could have mutations a RuvC domain, thereby rendering the nuclease domain non-functional.

A CRISPR system may be a Type VI CRISPR system, for example a C2c2 system. A Type VI nuclease may comprise a HEPN domain. In some examples, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some cases, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some examples, a Type VI nuclease may be catalytically dead such that it binds to a target sequence, but does not cleave. For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, homologues thereof, orthologues thereof, or modified versions thereof.

In some methods disclosed herein, Argonaute (Ago) systems may be used to cleave target nucleic acid sequences. Ago protein may be derived from a prokaryote, eukaryote, or archaea. The target nucleic acid may be RNA or DNA. A DNA target may be single stranded or double stranded. In some examples, the target nucleic acid does not require a specific target flanking sequence, such as a sequence equivalent to a protospacer adjacent motif or protospacer flanking sequence. The Ago protein may create a double strand break or single strand break. In some examples, when a Ago protein forms a single strand break, two Ago proteins may be used in combination to generate a double strand break. In some examples, an Ago protein comprises one, two, or more nuclease domains. In some examples, an Ago protein comprises one, two, or more catalytic domains. One or more nuclease or catalytic domains may be mutated in the Ago protein, thereby generating a nickase protein capable of generating single strand breaks. In other examples, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In many examples, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some examples, the gDNA may be less than 10 nucleotides in length. In some examples, the gDNA may be more than 50 nucleotides in length.

Argonaute-mediated cleavage may generate blunt end, 5′ overhangs, or 3′ overhangs. In some examples, one or more nucleotides are removed from the target site during or following cleavage.

Argonaute protein may be endogenously or recombinantly expressed. Argonaute may be encoded on a chromosome, extrachromosomally, or on a plasmid, synthetic chromosome, or artificial chromosome. Additionally or alternatively, an Argonaute protein may be provided as a polypeptide or mRNA encoding the polypeptide. In such examples, polypeptide or mRNA may be delivered through standard mechanisms known in the art, such as through the use of peptides, nanoparticles, or viral particles.

Guide DNAs may be provided by genetic or episomal DNA. In some examples, gDNA are reverse transcribed from RNA or mRNA. In some examples, guide DNAs may be provided or delivered concomitantly with an Ago protein or sequentially. Guide DNAs may be chemically synthesized, assembled, or otherwise generated using standard DNA generation techniques known in the art. Guide DNAs may be cleaved, released, or otherwise derived from genomic DNA, episomal DNA molecules, isolated nucleic acid molecules, or any other source of nucleic acid molecules.

Nuclease fusion proteins may be recombinantly expressed. A nuclease fusion protein may be encoded on a chromosome, extrachromosomally, or on a plasmid, synthetic chromosome, or artificial chromosome. A nuclease and a chromatin-remodeling enzyme may be engineered separately, and then covalently linked. A nuclease fusion protein may be provided as a polypeptide or mRNA encoding the polypeptide. In such examples, polypeptide or mRNA may be delivered through standard mechanisms known in the art, such as through the use of peptides, nanoparticles, or viral particles.

A guide nucleic acid (e.g., a gRNA) may complex with a compatible nucleic acid-guided nuclease and may hybridize with a target sequence, thereby directing the nuclease to the target sequence. A subject nucleic acid-guided nuclease capable of complexing with a guide nucleic acid may be referred to as a nucleic acid-guided nuclease that is compatible with the guide nucleic acid. Likewise, a guide nucleic acid capable of complexing with a nucleic acid-guided nuclease may be referred to as a guide nucleic acid, e.g., gRNA that is compatible with the nucleic acid-guided nucleases (e.g., a Cas enzyme molecule).

A guide nucleic acid may be DNA. A guide nucleic acid may be RNA. A guide nucleic acid may comprise both DNA and RNA. A guide nucleic acid may comprise modified of non-naturally occurring nucleotides. In cases where the guide nucleic acid comprises RNA, the RNA guide nucleic acid may be encoded by a DNA sequence on a polynucleotide molecule such as a plasmid, linear construct, or editing cassette as disclosed herein.

A guide nucleic acid may comprise a guide sequence. A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some aspects, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 nucleotides long. The guide sequence may be 10-25 nucleotides in length. The guide sequence may be 10-20 nucleotides in length. The guide sequence may be 15-30 nucleotides in length. The guide sequence may be 20-30 nucleotides in length. The guide sequence may be 15-25 nucleotides in length. The guide sequence may be 15-20 nucleotides in length. The guide sequence may be 20-25 nucleotides in length. The guide sequence may be 22-25 nucleotides in length. The guide sequence may be 15 nucleotides in length. The guide sequence may be 16 nucleotides in length. The guide sequence may be 17 nucleotides in length. The guide sequence may be 18 nucleotides in length. The guide sequence may be 19 nucleotides in length. The guide sequence may be 20 nucleotides in length. The guide sequence may be 21 nucleotides in length. The guide sequence may be 22 nucleotides in length. The guide sequence may be 23 nucleotides in length. The guide sequence may be 24 nucleotides in length. The guide sequence may be 25 nucleotides in length.

A guide nucleic acid may comprise a scaffold sequence. In general, a “scaffold sequence” includes any sequence that has sufficient sequence to promote formation of a targetable nuclease complex, wherein the targetable nuclease complex comprises a nucleic acid-guided nuclease and a guide nucleic acid comprising a scaffold sequence and a guide sequence. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some aspects, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some aspects, at least one of the two sequence regions is about or more than about 5, 6, 7, 8, 9, 10, 11, 12, 13, 14, 15, 16, 17, 18, 19, 20, 21, 22, 23, 24, 25, 30, 40, 50, or more nucleotides in length. In some aspects, at least one of the two sequence regions is about 10-30 nucleotides in length. At least one of the two sequence regions may be 10-25 nucleotides in length. At least one of the two sequence regions may be 10-20 nucleotides in length. At least one of the two sequence regions may be 15-30 nucleotides in length. At least one of the two sequence regions may be 20-30 nucleotides in length. At least one of the two sequence regions may be 15-25 nucleotides in length. At least one of the two sequence regions may be 15-20 nucleotides in length. At least one of the two sequence regions may be 20-25 nucleotides in length. At least one of the two sequence regions may be 22-25 nucleotides in length. At least one of the two sequence regions may be 15 nucleotides in length. At least one of the two sequence regions may be 16 nucleotides in length. At least one of the two sequence regions may be 17 nucleotides in length. At least one of the two sequence regions may be 18 nucleotides in length. At least one of the two sequence regions may be 19 nucleotides in length. At least one of the two sequence regions may be 20 nucleotides in length. At least one of the two sequence regions may be 21 nucleotides in length. At least one of the two sequence regions may be 22 nucleotides in length. At least one of the two sequence regions may be 23 nucleotides in length. At least one of the two sequence regions may be 24 nucleotides in length. At least one of the two sequence regions may be 25 nucleotides in length.

A scaffold sequence of a subject guide nucleic acid may comprise a secondary structure. A secondary structure may comprise a pseudoknot region. In some example, the compatibility of a guide nucleic acid and nucleic acid-guided nuclease is at least partially determined by sequence within or adjacent to a pseudoknot region of the guide RNA. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In aspects of the disclosure the terms “guide nucleic acid” refers to a polynucleotide comprising 1) a guide sequence capable of hybridizing to a target sequence and 2) a scaffold sequence capable of interacting with or complexing with a nucleic acid-guided nuclease as described herein.

A guide nucleic acid may be compatible with a nucleic acid-guided nuclease when the two elements may form a functional targetable nuclease complex capable of cleaving a target sequence. Often, a compatible scaffold sequence for a compatible guide nucleic acid may be found by scanning sequences adjacent to native nucleic acid-guided nuclease loci. In other words, native nucleic acid-guided nucleases may be encoded on a genome within proximity to a corresponding compatible guide nucleic acid or scaffold sequence.

Nucleic acid-guided nucleases may be compatible with guide nucleic acids that are not found within the nucleases endogenous host. Such orthogonal guide nucleic acids may be determined by empirical testing. Orthogonal guide nucleic acids may come from different bacterial species or be synthetic or otherwise engineered to be non-naturally occurring.

Orthogonal guide nucleic acids that are compatible with a common nucleic acid-guided nuclease may comprise one or more common features. Common features may include sequence outside a pseudoknot region. Common features may include a pseudoknot region. Common features may include a primary sequence or secondary structure.

A guide nucleic acid may be engineered to target a desired target sequence by altering the guide sequence such that the guide sequence is complementary to the target sequence, thereby allowing hybridization between the guide sequence and the target sequence. A guide nucleic acid with an engineered guide sequence may be referred to as an engineered guide nucleic acid. Engineered guide nucleic acids are often non-naturally occurring and are not found in nature.

In some aspects the guide RNA molecule interferes with sequencing directly, for example by binding the target sequence to prevent nucleic acid polymerization to occur across the bound sequence. In some aspects the guide RNA molecule works in tandem with a RNA-DNA hybrid binding moiety such as a protein. In some aspects the guide RNA molecule directs modification of member of the sequencing library to which it may bind, such as methylation, base excision, or cleavage, such that in some aspects the member of the sequencing library to which it is bound becomes unsuitable for further sequencing reactions. In some aspects, the guide RNA molecule directs endonucleolytic cleavage of the DNA molecule to which it is bound, for example by a protein having endonuclease activity such as Cas9 protein. Zinc Finger Nucleases (ZFN), Transcription activator like effector nucleases and Clustered Regulatory Interspaced Short Palindromic Repeat/Cas based RNA guided DNA nuclease (CRISPR/Cas9), among others, are compatible with some aspects of the disclosure herein.

A guide RNA molecule comprises sequence that base-pairs with target sequence that is to be removed from sequencing (the first nucleic acid). In some aspects the base-pairing is complete, while in some aspects the base pairing is partial or comprises bases that are unpaired along with bases that are paired to non-target sequence.

A guide RNA may comprise a region or regions that form an RNA ‘hairpin’ structure. Such region or regions comprise partially or completely palindromic sequence, such that 5′ and 3′ ends of the region may hybridize to one another to form a double-strand ‘stem’ structure, which in some aspects is capped by a non-palindromic loop tethering each of the single strands in the double strand loop to one another.

In some aspects, the Guide RNA comprises a stem loop such as a tracrRNA stem loop. A stem loop such as a tracrRNA stem loop may complex with or bind to a nucleic acid endonuclease such as Cas9 endonuclease. Alternately, a stem loop may complex with an endonuclease other than Cas9 or with a nucleic acid modifying enzyme other than an endonuclease, such as a base excision enzyme, a methyltransferase, or an enzyme having other nucleic acid modifying activity that interferes with one or more DNA polymerase enzymes.

The tracrRNA/CRISPR/Endonuclease system was identified as an adaptive immune system in eubacterial and archaeal prokaryotes whereby cells gain resistance to repeated infection by a virus of a known sequence. See, for example, Deltcheva E, Chylinski K, Sharma C M, Gonzales K, Chao Y, Pirzada Z A et al. (2011) “CRISPR RNA maturation by trans-encoded small RNA and host factor RNase III” Nature 471 (7340): 602-7. doi: 10.1038/nature09886. PMC 3070239. PMID 21455174; Terns M P, Terns R M (2011) “CRISPR-based adaptive immune systems” Curr Opin Microbiol 14 (3): 321-7. doi: 10.1016/j.mib.2011.03.005. PMC 3119747. PMID 21531607; Jinek M, Chylinski K, Fonfara I, Hauer M, Doudna J A, Charpentier E (2012) “A Programmable Dual-RNA-Guided DNA Endonuclease in Adaptive Bacterial Immunity” Science 337 (6096): 816-21. doi: 10.1126/science.1225829. PMID 22745249; and Brouns S J (2012) “A swiss army of Science knife immunity” 337 (6096): 808-9. doi: 10.1126/science.1227253. PMID 22904002. The system has been adapted to direct targeted mutagenesis in eukaryotic cells. See, e.g., Wenzhi Jiang, Huanbin Zhou, Honghao Bi, Michael Fromm, Bing Yang, and Donald P. Weeks (2013) “Demonstration of CRISPR/Cas9/sgRNA-mediated targeted gene modification in Arabidopsis, tobacco, sorghum and rice” Nucleic Acids Res. November 2013; 41 (20): e188, Published online Aug. 31, 2013. doi: 10.1093/nar/gkt780, and references therein.

As contemplated herein, guide RNA are used in some aspects to provide sequence specificity to a DNA endonuclease such as a Cas9 endonuclease. In these aspects a guide RNA comprises a hairpin structure that binds to or is bound by an endonuclease such as Cas9 (other endonucleases are contemplated as alternatives or additions in some aspects), and a guide RNA further comprises a recognition sequence that binds to or specifically binds to or exclusively binds to a sequence that is to be removed from a sequencing library or a sequencing reaction. The length of the recognition sequence in a guide RNA may vary according to the degree of specificity desired in the sequence elimination process. Short recognition sequences, comprising frequently occurring sequence in the sample or comprising differentially abundant sequence (abundance of AT in an AT-rich genome sample or abundance of GC in a GC-rich genome sample) are likely to identify a relatively large number of sites and therefore to direct frequent nucleic acid modification such as endonuclease activity, base excision, methylation or other activity that interferes with at least one DNA polymerase activity. Long recognition sequences, comprising infrequently occurring sequence in the sample or comprising underrepresented base combinations (abundance of GC in an AT-rich genome sample or abundance of AT in a GC-rich genome sample) are likely to identify a relatively small number of sites and therefore to direct infrequent nucleic acid modification such as endonuclease activity, base excision, methylation or other activity that interferes with at least one DNA polymerase activity. Accordingly, as disclosed herein, in some aspects one may regulate the frequency of sequence removal from a sequence reaction through modifications to the length or content of the recognition sequence.

Guide RNA may be synthesized through a number of methods consistent with the disclosure herein. Standard synthesis techniques may be used to produce massive quantities of guide RNAs, and/or for highly-repetitive targeted regions, which may require only a few guide RNA molecules to target a multitude of unwanted loci. The double stranded DNA molecules can comprise an RNA site specific binding sequence, a guide RNA sequence for Cas9 protein and a T7 promoter site. In some cases, the double stranded DNA molecules can be less than about 100 bp length. T7 polymerase can be used to create the single stranded RNA molecules, which may include the target RNA sequence and the guide RNA sequence for the Cas9 protein.

Guide RNA sequences may be designed through a number of methods. For example, In some aspects, non-genic repeat sequences of the human genome are broken up into, for example, 100 bp sliding windows. Double stranded DNA molecules can be synthesized in parallel on a microarray using photolithography.

The windows may vary in size. 30-mer target sequences can be designed with a short trinucleotide protospacer adjacent motif (PAM) sequence of N-G-G flanking the 5′ end of the target design sequence, which in some cases facilitates cleavage. See, among others, Giedrius Gasiunas et al., (2012) “Cas9-crRNA ribonucleoprotein complex mediates specific DNA cleavage for adaptive immunity in bacteria” Proc. Natl. Acad. Sci. USA. September 25, 109(39): E2579-E2586, which is hereby incorporated by reference in its entirety. Redundant sequences can be eliminated and the remaining sequences can be analyzed using a search engine (e.g. BLAST) against the human genome to avoid hybridization against REFSEQ, ENSEMBL and other gene databases to avoid nuclease activity at these sites. The universal Cas9 tracer RNA sequence can be added to the guide RNA target sequence and then flanked by the T7 promoter. The sequences upstream of the T7 promoter site can be synthesized. Due to the highly repetitive nature of the target regions in the human genome, in many aspects, a relatively small number of guide RNA molecules will digest a larger percentage of NGS library molecules.

Although only about 50% of protein coding genes are estimated to have exons comprising the NGG PAM (photospacer adjacent motif) sequence, multiple strategies are provided herein to increase the percentage of the genome that can be targeted with the Cas9 cutting system. For example, if a PAM sequence is not available in a DNA region, a PAM sequence may be introduced via a combination strategy using a guide RNA coupled with a helper DNA comprising the PAM sequence. The helper DNA can be synthetic and/or single stranded. The PAM sequence in the helper DNA will not be complimentary to the gDNA knockout target in the NGS library, and may therefore be unbound to the target NGS library template, but it can be bound to the guide RNA. The guide RNA can be designed to hybridize to both the target sequence and the helper DNA comprising the PAM sequence to form a hybrid DNA: RNA: DNA complex that can be recognized by the Cas9 system.

The PAM sequence may be represented as a single stranded overhang or a hairpin. The hairpin can, in some cases, comprise modified nucleotides that may optionally be degraded. For example, the hairpin can comprise Uracil, which can be degraded by Uracil DNA Glycosylase.

As an alternative to using a DNA comprising a PAM sequence, modified Cas9 proteins without the need of a PAM sequence or modified Cas9 with lower sensitivity to PAM sequences may be used without the need for a helper DNA sequence.

In further cases, the guide RNA sequence used for Cas9 recognition may be lengthened and inverted at one end to act as a dual cutting system for close cutting at multiple sites. The guide RNA sequence can produce two cuts on a NGS DNA library target. This can be achieved by designing a single guide RNA to alternate strands within a restricted distance. One end of the guide RNA may bind to the forward strand of a double stranded DNA library and the other may bind to the reverse strand. Each end of the guide RNA can comprise the PAM sequence and a Cas9 binding domain. This may result in a dual double stranded cut of the NGS library molecules from the same DNA sequence at a defined distance apart.

Alternative versions of the assay comprise at least one sequence-specific nuclease, and in some cases a combination of sequence-specific nucleases, such as at least one restriction endonuclease having a recognition site that is abundant in the first nucleic acid. In some cases an enzyme comprises an activity that yields double-stranded breaks in response to a specific sequence. In some cases an enzyme comprises any nuclease or other enzyme that digests double-stranded nucleic acid material in RNA/DNA hybrids.

Nucleic acid probes (e.g. biotinylated probes) complementary to the second nucleic acids can be hybridized to the second nucleic acids in solution and pulled down with, e.g., magnetic streptavidin-coated beads. Non bound nucleic acids can be washed away and the captured nucleic acids may then be eluted and amplified for sequencing or genotyping.

In some aspects, practice of the methods herein reduces the sequencing time duration of a sequencing reaction, such that a nucleic acid library is sequenced in a shorter time, or using fewer reagents, or using less computing power. In some aspects, practice of the methods herein reduces the sequencing time duration of a sequencing reaction for a given nucleic acid library to about 90%, 80%, 70%, 60%, 50%, 40%, 33%, 30% or less than 30% of the time required to sequence the library in the absence of the practice of the methods herein.

In some aspects, a specific read sequence from a specific region is of particular interest in a given sequencing reaction. Measures to allow the rapid identification of such a specific region are beneficial as they may decrease computation time or reagent requirements or both computation time and reagent requirements.

Some aspects relate to the generation of guide RNA molecules. Guide RNA molecules are in some cases transcribed from DNA templates. A number of RNA polymerases may be used, such as T7 polymerase, RNA Poll, RNA PolII, RNA PolIII, an organellar RNA polymerase, a viral RNA polymerase, or a eubacterial or archaeal polymerase. In some cases the polymerase is T7.

Guide RNA generating templates comprise a promoter, such as a promoter compatible with transcription directed by T7 polymerase, RNA Poll, RNA PolII, RNA PolIII, an organellar RNA polymerase, a viral RNA polymerase, or a eubacterial or archaeal polymerase. In some cases the promoter is a T7 promoter.

Guide RNA templates encode a tag sequence in some cases. A tag sequence binds to a nucleic acid modifying enzyme such as a methylase, base excision enzyme or an endonuclease. In the context of a larger Guide RNA molecule bound to a nontarget site, a tag sequence tethers an enzyme to a nucleic acid nontarget region, directing activity to the nontarget site. An exemplary tethered enzyme is an endonuclease such as Cas9.

Guide RNA templates are complementary to the first nucleic acid corresponding to ribosomal RNA sequences, sequences encoding globin proteins, sequences encoding a transposon, sequences encoding retroviral sequences, sequences comprising telomere sequences, sequences comprising sub-telomeric repeats, sequences comprising centromeric sequences, sequences comprising intron sequences, sequences comprising Alu repeats, sequences comprising SINE repeats, sequences comprising LINE repeats, sequences comprising dinucleic acid repeats, sequences comprising trinucleic acid repeats, sequences comprising tetranucleic acid repeats, sequences comprising poly-A repeats, sequences comprising poly-T repeats, sequences comprising poly-C repeats, sequences comprising poly-G repeats, sequences comprising AT-rich sequences, or sequences comprising GC-rich sequences.

In many cases, the tag sequence comprises a stem-loop, such as a partial or total stem-loop structure. The ‘stem’ of the stem loop structure is encoded by a palindromic sequence in some cases, either complete or interrupted to introduce at least one ‘kink’ or turn in the stem. The ‘loop’ of the stem loop structure is not involved in stem base pairing in most cases. In some cases, the stem loop is encoded by a tracr sequence, such as a tracr sequence disclosed in references incorporated herein. Some stem loops bind, for example, Cas9 or other endonuclease.

Guide RNA molecules additionally comprise a recognition sequence. The recognition sequence is completely or incompletely reverse-complementary to a nontarget sequence to be eliminated from a nucleic acid library sequence set. As RNA is able to hybridize using base pair combinations (G: U base pairing, for example) that do not occur in DNA-DNA hybrids, the recognition sequence does not need to be an exact reverse complement of the nontarget sequence to bind. In addition, small perturbations from complete base pairing are tolerated in some cases.

End protection: Protecting the ends of DNA molecules from degradation can be effected through a number of approaches, provided that an end result is prevention of adapter-added fragments from exonuclease degradation at the site of adapter attachment. Adapters are added through ligation, polymerase mediated amplification, tagmentation via transposase delivery, end modification or other approaches. Representative adapters include hairpin adapters that effectively link the two strands of a double-stranded nucleic acid to form a single-stranded circular molecule if added at both ends. Such a molecule lacks an exposed end for single stranded or double stranded exonuclease degradation unless it is further cleaved by an endonuclease. Protection is also effected by attachment of an oligonucleotide or other molecule that is resistant to exonuclease activity. Examples of exonuclease-resistant adapters include phosphorthioate oligos, 2-O methyl modified nucleotide sugars, inverted dT or ddT, phosphorylation, C3 spacers or other modifications that inhibit an exonuclease from traversing the modification so as do degrade adjacent nucleic acids. Alternately or in combination, in some cases an ‘adapter’ constitutes modification to the ends of sample nucleic acids without ligation of additional molecules, such that the modification renders the nucleic acids resistant to exonuclease degradation.

A particular feature of the adapters herein is that, although they operate locally independent of one another, a nucleic acid is not protected from degradation unless both ends are subjected to adapter addition or modification. Otherwise, although and adapter-added end is protected from exonuclease activity, the opposite end of the nucleic acid is vulnerable to degradation such that the molecule as a whole is degraded. This is the fate of nucleic acids that are adapter modified but then cleaved by a sequence-specific nucleic acid endonuclease as contemplated herein, so as to yield at least two exposed, unprotected nucleic acid ends.

### Non-Host Nucleic Acids

Targeted depletion methods herein result in removal of a first nucleic acid and enrichment of a second nucleic acid from the sample. Said sample can be used to make a library for sequencing and said sequencing delivers sequence data that can be mostly derived from the second nucleic acid. For example, the second nucleic acid can be a non-host nucleic acid.

In certain aspects, provided herein are methods that result in enrichment of a microbial pathogen. In some cases, methods herein enable identification of said microbial pathogen. In some aspects the microbial pathogen comprises a bacterial pathogen. In some aspects, the bacterial pathogen is a Bacillus such as a Bacillus anthracis or a Bacillus cereus; a Bartonella such as a Bartonella henselae or a Bartonella quintana; a Bordetella such as a Bordetella pertussis; a Borrelia such as a Borrelia burgdorferi, a Borrelia garinii, a Borrelia afzelii, a Borrelia recurrentis; a Brucella such as a Brucella abortus, a Brucella canis, a Brucella melitensis or a Brucella suis; a Campylobacter such as a Campylobacter jejuni; a Chlamydia or Chlamydophila such as Chlamydia pneumoniae, Chlamydia trachomatis, Chlamydophila psittaci; a Clostridium such as a Clostridium botulinum, a Clostridium difficile, a Clostridium perfringens, a Clostridium tetani; a Corynebacterium such as a Corynebacterium diphtheriae; an Enterococcus such as a Enterococcus faecalis or a Enterococcus faecium; a Escherichia such as a Escherichia coli; a Francisella such as a Francisella tularensis; a Haemophilus such as a Haemophilus influenzae; a Helicobacter such as a Helicobacter pylori; a Legionella such as a Legionella pneumophila; a Leptospira such as a Leptospira interrogans, a Leptospira santarosai, a Leptospira weilii or a Leptospira noguchii; a Listeria such as a Listeria monocytogenes; a Mycobacterium such as a Mycobacterium leprae, a Mycobacterium tuberculosis or a Mycobacterium ulcerans; a Mycoplasma such as a Mycoplasma pneumoniae; a Neisseria such as a Neisseria gonorrhoeae or a Neisseria meningitidis; a Pseudomonas such as a Pseudomonas aeruginosa; a Rickettsia such as a Rickettsia rickettsii; a Salmonella such as a Salmonella typhi or a Salmonella typhimurium; a Shigella such as a Shigella sonnei; a Staphylococcus such as a Staphylococcus aureus, a Staphylococcus epidermidis, a Staphylococcus saprophyticus; a Streptococcus such as a Streptococcus agalactiae, a Streptococcus pneumoniae, a Streptococcus pyogenes; a Treponema such as a Treponema pallidum; a Vibrio such as a Vibrio cholerae; a Yersinia such as a Yersinia pestis, a Yersinia enterocolitica or a Yersinia pseudotuberculosis. In some aspects, the microbial pathogen comprises a viral pathogen. In some aspects, the viral pathogen comprises a Adenoviridae such as, an Adenovirus; a Herpesviridae such as a Herpes simplex, type 1, a Herpes simplex, type 2, a Varicella-zoster virus, an Epstein-barr virus, a Human cytomegalovirus, a Human herpesvirus, type 8; a Papillomaviridae such as a Human papillomavirus; a Polyomaviridae such as a BK virus or a JC virus; a Poxviridae such as a Smallpox; a Hepadnaviridae such as a Hepatitis B virus: a Parvoviridae such as a Human bocavirus or a Parvovirus; a Astroviridae such as a Human astrovirus; a Caliciviridae such as a Norwalk virus; a Picornaviridae such as a coxsackievirus, a hepatitis A virus, a poliovirus, a rhinovirus; a Coronaviridae such as a Severe acute respiratory syndrome virus or a Wuhan coronavirus; a Flaviviridae such as a Hepatitis C virus, a yellow fever virus, a dengue virus, a West Nile virus; a Togaviridae such as a Rubella virus; a Hepeviridae such as a Hepatitis E virus; a Retroviridae such as a Human immunodeficiency virus (HIV); a Orthomyxoviridae such as an Influenza virus; a Arenaviridae such as a Guanarito virus, a Junin virus, a Lassa virus, a Machupo virus, a Sabiá virus; a Bunyaviridae such as a Crimean-Congo hemorrhagic fever virus; a Filoviridae such as a Ebola virus, a Marburg virus; a Paramyxoviridae such as a Measles virus, a Mumps virus, a Parainfluenza virus, a Respiratory syncytial virus, a Human metapneumovirus, a Hendra virus, a Nipah virus; a Rhabdoviridae such as a Rabies virus; a Hepatitis D virus; or a Reoviridae such as a Rotavirus, a Orbivirus, a Coltivirus, a Banna virus pathogen. In some aspects, the microbial pathogen comprises a fungal pathogen. In some aspects, the fungal pathogen comprises actinomycosis, allergic bronchopulmonary aspergillosis, aspergilloma, aspergillosis, athlete's foot, basidiobolomycosis, Basidiobolus ranarum, black piedra, blastomycosis, Candida krusei, candidiasis, chronic pulmonary aspergillosis, chrysosporium, chytridiomycosis, coccidioidomycosis, conidiobolomycosis, cryptococcosis, Cryptococcus gattii, deep dermatophytosis, dermatophyte, dermatophytid, dermatophytosis, endothrix, entomopathogenic fungus, epizootic lymphangitis, esophageal candidiasis, exothrix, fungal meningitis, fungemia, geotrichum, Geotrichum candidum, histoplasmosis, lobomycosis, Massospora cicadina, Microsporum gypseum, muscardine, mycosis, myringomycosis, neozygites remaudierei, Neozygites slavi, Ochroconis gallopava, Ophiocordyceps arborescens, Ophiocordyceps coenomyia, Ophiocordyceps macroacicularis, Ophiocordyceps nutans, oral candidiasis, paracoccidioidomycosis, pathogenic dimorphic fungi, penicilliosis, piedra, piedraia, pneumocystis pneumonia, pseudallescheriasis, scedosporiosis, sporotrichosis, Tinea, Tinea barbae, Tinea capitis, Tinea corporis, Tinea cruris, Tinea faciei, Tinea incognito, Tinea nigra, Tinea pedis, Tinea versicolor, vomocytosis, white nose syndrome, zeaspora, or zygomycosis. In some cases, methods herein result in enrichment of a protozoon nucleic acid. In some cases, methods herein result in enrichment of a cancer nucleic acid. In some cases, methods herein result in enrichment of a fetal nucleic acid.

### Use of Endonuclease/Exonuclease Combinations in Targeted Depletion

The method described herein for depleting a first nucleic acid may result in a sequencing library with dramatically reduced complexity. Unwanted sequences are removed and the remaining sequences can be more readily analyzed by NGS techniques. The reduced complexity of the library can reduce the sequencer capacity required for clinical depth sequencing and/or reduce the computational requirement for accurate mapping of non-repetitive sequences. The sequence that is enriched can be searched in a bioinformatics database such as BLAST to determine the identity of the genes. The sequence information of the enriched nucleic acid can be used to determine the type of pathogen.

Methods described herein can include performing a genetic analysis of the second nucleic acid (e.g., enriched nucleic acid). Genome sequence databases can be searched to find sequences which are related to the second nucleic acid. The search can generally be performed by using computer-implemented search algorithms to compare the query sequences with sequence information stored in a plurality of databases accessible via a communication network, for example, the Internet. Examples of such algorithms include the Basic Local Alignment Search Tool (BLAST) algorithm, the PSI-blast algorithm, the Smith-Waterman algorithm, the Hidden Markov Model (HMM) algorithm, and other like algorithms.

In some aspects, the endonuclease is configured such that it targets a plurality of sites in the genome to be depleted; thereafter, exonuclease digestion generates nucleic acid molecules or fragments that can be excluded from the nucleic acid molecules that are ligated to the adapters, cloned and prepared a library from.

Hence, provided herein is an improved method of preparing a library comprising selective nucleic acid molecules from a sample comprising a first nucleic acid and a second nucleic acid, comprising: providing a sample comprising the first nucleic acid and a second nucleic acid; subjecting the sample to a process that removes a nucleic acid fragment that is less than a threshold size from the sample; subjecting the first nucleic acid and the second nucleic acid to an endonuclease to form at least one cleaved first nucleic acid, wherein the endonuclease cleaves the first nucleic acid but does not cleave the second nucleic acid; contacting the sample from step (c) to an exonuclease generating exonuclease digested nucleic acid molecules; enriching the exonuclease digested nucleic acid molecules that are greater than the threshold size and generating a library comprising the enriched nucleic acid molecules.

In some aspects, provided herein is an improved method for enriching selective nucleic acid molecules, such as a contaminated sample or a biological sample. In some aspects, the methods provided herein increases the specificity of the enriched nucleic acid. In some aspects, the method comprises an additional step of size exclusion cleaning and enrichments. In some aspects, the methods provided herein increases the yield of the enriched nucleic acid. In some aspects the method comprises elimination of a purification step for higher yield.

In some aspects, the yield is increased by 10%, 20%, 30%, 40%, 50%, 60%, 70%, 80%, 90% or 100% or more compared to conventional methods.

Definitions: A partial list of relevant definitions is as follows.

In some cases, the term “enriched” may be used in a relative sense, such that a second nucleotide or population comprising a second nucleotide is enriched upon the selective depletion of a first nucleotide or population comprising a first nucleotide. It may not need increase in an absolute sense to be enriched. Rather, an absolute increase or a relative increase resulting from depletion or deletion of other nucleic acids may constitute ‘enrichment’ as used herein.

As used herein, the term “deplete” or “depleting” may be used in a relative sense, such that a first nucleotide or population comprising a first nucleotide is degraded upon the selective preservation of a second nucleotide or population comprising a second nucleotide. It may not need decrease in an absolute sense to be depleted. Rather, an absolute decrease or a relative decrease resulting from preservation of other nucleic acids may constitute ‘depleting’ as used herein.

As used herein, “about” a given value is defined as +/−10% of said given value.

As used herein, NGS or Next Generation Sequencing may refer to any number of nucleic acid sequencing technologies, such as 5.1 Massively parallel signature sequencing (MPSS), Polony sequencing, 454 pyrosequencing, Illumina (Solexa) sequencing, SOLID sequencing, Ion Torrent semiconductor sequencing, DNA nanoball sequencing, Heliscope single molecule sequencing, Single molecule real time (SMRT) sequencing, Tunnelling currents DNA sequencing, Sequencing by hybridization, Sequencing with mass spectrometry, Microfluidic Sanger sequencing, Microscopy-based techniques, RNAP sequencing, and In vitro virus high-throughput sequencing.

As used herein, to ‘modify’ a nucleic acid may be to cause a change to a covalent bond in the nucleic acid, such as methylation, base removal, or cleavage of a phosphodiester backbone.

As used herein, to ‘direct transcription’ may be to provide template sequence from which a specified RNA molecule can be transcribed.

“Amplified nucleic acid” or “amplified polynucleotide” includes any nucleic acid or polynucleotide molecule whose amount has been increased by any nucleic acid amplification or replication method performed in vitro as compared to its starting amount. For example, an amplified nucleic acid is optionally obtained from a polymerase chain reaction (PCR) which can, in some instances, amplify DNA in an exponential manner (for example, amplification to 2n copies in n cycles) wherein most products are generated from intermediate templates rather than directly from the sample template. Amplified nucleic acid is alternatively obtained from a linear amplification, where the amount increases linearly over time and which, in some cases, produces products that are synthesized directly from the sample.

The term “biological sample” or “sample” generally refers to a sample or part isolated from a biological entity. The biological sample, in some cases, shows the nature of the whole biological entity and examples include, without limitation, bodily fluids, dissociated tumor specimens, cultured cells, and any combination thereof. Biological samples come from one or more individuals. One or more biological samples come from the same individual. In one non limiting example, a first sample is obtained from an individual's blood and a second sample is obtained from an individual's tumor biopsy. Examples of biological samples include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, breath, hair, finger nails, skin, biopsy, placental fluid, amniotic fluid, cord blood, emphatic fluids, cavity fluids, sputum, pus, microbiota, meconium, breast milk and/or other excretions. In some cases, a blood sample comprises circulating tumor cells or cell free DNA, such as tumor DNA or fetal DNA. The samples include nasopharyngeal wash. Examples of tissue samples of the subject include but are not limited to, connective tissue, muscle tissue, nervous tissue, epithelial tissue, cartilage, cancerous or tumor sample, or bone. Samples are obtained from a human or an animal. Samples are obtained from a mammal, including vertebrates, such as murines, simians, humans, farm animals, sport animals, or pets. Samples are obtained from a living or dead subject. Samples are obtained fresh from a subject or have undergone some form of pre-processing, storage, or transport.

Nucleic acid sample as used herein refers to a nucleic acid sample for which the first nucleic acid is to be determined, A nucleic acid sample is extracted from a biological sample above, in some cases. Alternatively, a nucleic acid sample is artificially synthesized, synthetic, or de novo synthesized in some cases. The DNA sample is genomic in some cases, while in alternate cases the DNA sample is derived from a reverse-transcribed RNA sample.

“Bodily fluid” generally describes a fluid or secretion originating from the body of a subject. In some instances, bodily fluid is a mixture of more than one type of bodily fluid mixed together. Some non-limiting examples of bodily fluids include but are not limited to: blood, urine, bone marrow, spinal fluid, pleural fluid, lymphatic fluid, amniotic fluid, ascites, sputum, or a combination thereof.

“Complementary” or “complementarity,” or, in some cases more accurately “reverse-complementarity” refer to nucleic acid molecules that are related by base-pairing. Complementary nucleotides are, generally, A and T (or A and U), or C and G (or G and U). Functionally, two single stranded RNA or DNA molecules are complementary when they form a double-stranded molecule through hydrogen-bond mediated base paring. Two single stranded RNA or DNA molecules are said to be substantially complementary when the nucleotides of one strand, optimally aligned and with appropriate nucleotide insertions or deletions, pair with at least about 90% to about 95% or greater complementarity, and more preferably from about 98% to about 100%) complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions and not stringent hybridization conditions. Hybridization temperatures are generally at least about 2° C. to about 6° C. lower than melting temperatures (Tm).

“Double-stranded” or “double stranded” refers, in some cases, to two polynucleotide strands that have annealed through complementary base-pairing, such as in a reverse-complementary orientation.

“Known oligonucleotide sequence” or “known oligonucleotide” or “known sequence” may refer to a polynucleotide sequence that is known. In some cases, a known oligonucleotide sequence corresponds to an oligonucleotide that has been designed, e.g., a universal primer for next generation sequencing platforms (e.g., Illumina, 454), a probe, an adapter, a tag, a primer, a molecular barcode sequence, an identifier. A known sequence optionally comprises part of a primer. A known oligonucleotide sequence, in some cases, may not actually be known by a particular user but is constructively known, for example, by being stored as data accessible by a computer. A known sequence may optionally be a trade secret that is actually unknown or a secret to one or more users but is known by the entity who has designed a particular component of the experiment, kit, apparatus or software that the user is using.

“Library” in some cases may refer to a collection of nucleic acids. A library optionally contains one or more target fragments. In some instances the target fragments comprise amplified nucleic acids. In other instances, the target fragments comprise nucleic acid that is not amplified. A library optionally contains nucleic acid that has one or more known oligonucleotide sequence(s) added to the 3′ end, the 5′ end or both the 3′ and 5′ end. The library is optionally prepared so that the fragments contain a known oligonucleotide sequence that identifies the source of the library (e.g., a molecular identification barcode identifying a patient or nucleic acid material source). In some instances, two or more libraries are pooled to create a library pool. Libraries are optionally generated with other kits and techniques such as transposon mediated labeling, or “tagmentation” as known in the art. Kits are commercially available. One non-limiting example of a kit is the Illumina NEXTERA kit (Illumina, San Diego, CA).

The term “polynucleotides” or “nucleic acids” includes but is not limited to various DNA, RNA molecules, derivatives or combination thereof. These include species such as dNTPs, ddNTPs, DNA, RNA, peptide nucleic acids, cDNA, dsDNA, ssDNA, plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA.

Before the present methods, compositions and kits are described in greater detail, it is to be understood that this invention is not limited to particular method, composition or kit described, as such may, of course, vary. It is also to be understood that the terminology used herein is for the purpose of describing particular aspects only, and is not intended to be limiting, since the scope of the present invention will be limited only by the appended claims as construed herein. Examples are put forth so as to provide those of ordinary skill in the art with a more complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Integration of library preparation method into system for automatic processing of the reaction: In order to integrate the methods of sample processing to sequencing and diagnosis of a target sequence, or to generate a library in a bench to bedside workflow a device may be designed, that can perform liquid handling and transfers in a programmable sequence. The method described above is designed for minimizing liquid transfer steps, and multiple steps can be performed in a single vessel eliminating the need for vessel to vessel transfer of samples. Several systems exist that are commercially available for hands free sequencing of samples. Exemplary NGS capable systems may include EpMotion® 5075t Automated Liquid Handler; Perkin Elmer JANUS NGS Express AJS4NGS/D Automated Liquid Handler; and others.

Systems may be available that integrate liquid handling in NGS workflow from the extraction phase to the sequencing reaction. Exemplary systems include but are not limited to the Rhoenix workstation.

With the method optimized for minimized clean up and transfer stapes, it can be integrated into an automatic system devised or further optimized from the available systems for hands free, scalable workflow suitable for “sample to answer” reactions in clinical POC settings.

Unless defined otherwise, all technical and scientific terms used herein have the same meaning as commonly understood by one of ordinary skill in the art to which this invention belongs. Although any methods and materials similar or equivalent to those described herein are optionally used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method is contemplated to be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

In some aspects, the method described herein comprises the following steps: a nucleic acid sample is depleted of DNA molecules that are relatively small, such as less than 1 kb, (ii) the genomic nucleic acid to be depleted, such as human genomic nucleic acid, may be digested to fragment sizes less than 1 kb, (iii) the digested nucleic acids are sorted and selected based on size and (iv) a library is made from the selected digested material. This can be done on genomic DNA as well as full length cDNA.

The descriptions discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are given for the purpose of illustrating various aspects of the invention and are not meant to limit the present invention in any fashion. The present examples, along with the methods described herein are presently representative of preferred aspects, are exemplary, and are not intended as limitations on the scope of the invention. Changes therein and other uses which are encompassed within the spirit of the invention as defined by the scope of the claims will occur to those skilled in the art.

### Example 1. Generation of 5′ and 3′ Adapter Ligated Single Stranded cDNA

Provided herein is a detailed method for preparing a 5′ and 3′ adapter ligated cDNA first strand—for fast and efficient generation of an amplified cDNA library.

Following isolation of total RNA from a sample and fragmentation, a first strand cDNA synthesis is performed with random primer having an universal adapter sequence at its 5′ end. Usual precautions are taken for protecting the reaction from RNase action. The reaction is subjected to 50° C. temperature, in which it is incubated for about 30-60 minutes, as determined by one of skill in the art. The reverse transcriptase reaction is completed with a brief incubation to 75° C., for about 10 minutes.

At the end of this reaction, Exo 1 (exonuclease) and Shrimp Alkaline phosphatase (rSAP) are added to the reaction mixture. The primers are digested by addition of exonuclease 1. dNTPs are digested with rSAP so that they do not interfere with Terminal deoxynucleotidyl Transferase (TdT) tailing reaction next. All these reactions can be performed serially without the need for purifying the samples, and running through centrifugation columns.

In the next step the cDNA is denatured from the RNA by heating at 95° C. for 15 minutes, which also deactivates the SAP and Exo 1. The Mg++ present in the buffer from the initial fragmentation step with continue to chew the RNA fragment now denatured. The intact cDNA strand is available for TdT reaction. A mixture of ribonucleotides (NTPs) is added to the solution. TdT enzyme solution is added along with requisite amount of TdT buffer. No Cobalt-containing buffer is added. The reaction is allowed to proceed at 37° C. for 30 minutes. 1-4 ribonucleotides are added at the 3′ end. Preferably the ribonucleotides are not Guanine repeats. Random incorporation of 1, 2, 3 and 4 ribonucleotides can generate UMI codes for each cDNA.

To this reaction, adapter ligation reagents are added after the incubation period. An App-B adenylated DNA oligonucleotide adapter is ligated to the 3′terminal ribonucleotide. T4 RNA ligase 2 truncated enzyme is used for ligation in the presence of about 15% PEG8000. Ligation is carried out at the manufacturer's recommended temperature, e.g., 28° C. for 30 minutes.

Finally, following ligation, the products are captured with Ampure XP beads (1.8×) by adding the bead solution in the preexisting ligation mixture and incubating for a period of time to allow binding RNA/cDNA at room temperature, beads are washed 2 times with 150 μL 80% EtOH, air dried and eluted with 24 μL 10 mM Tris pH 8. The eluant is transferred to fresh PCR tube.

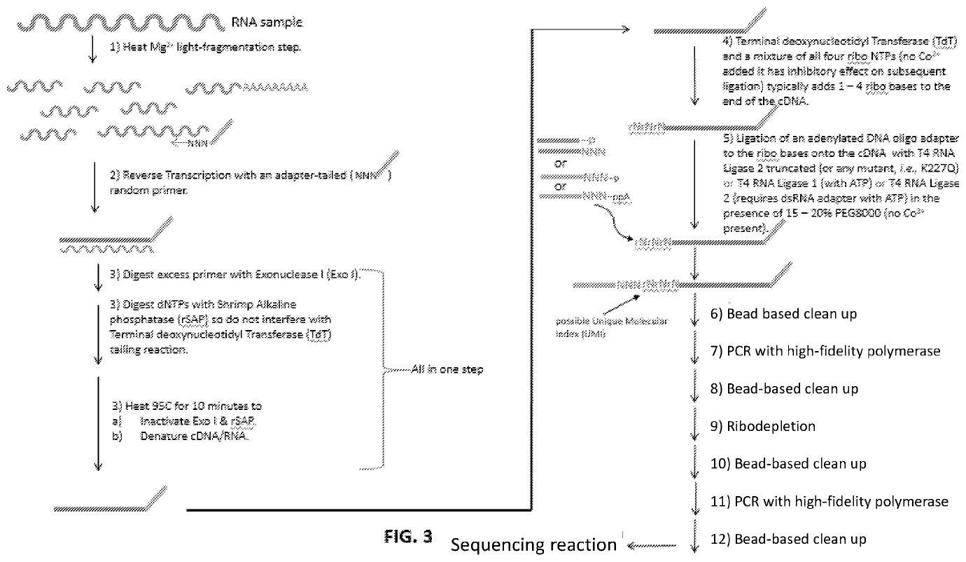

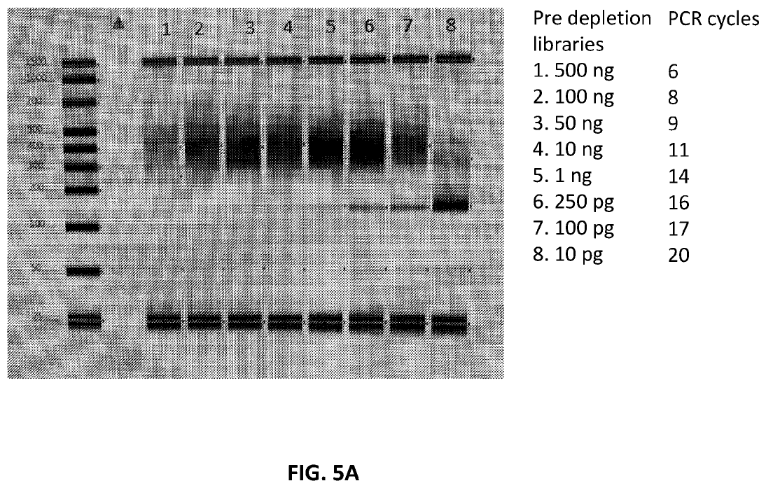

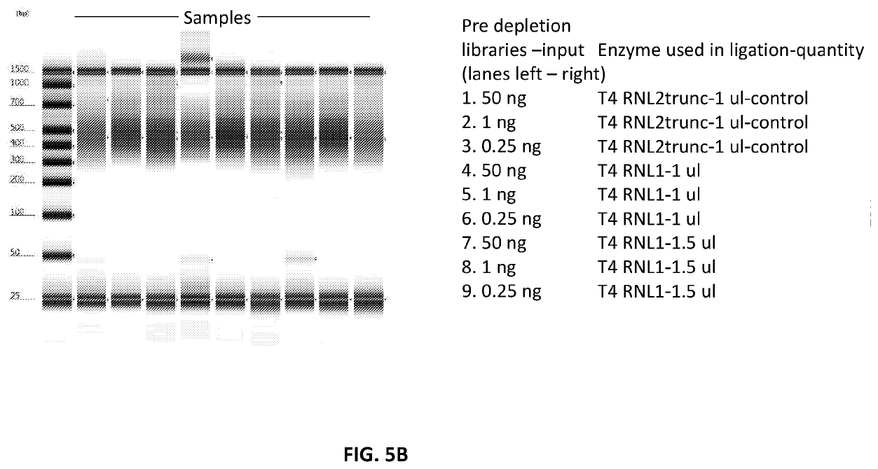

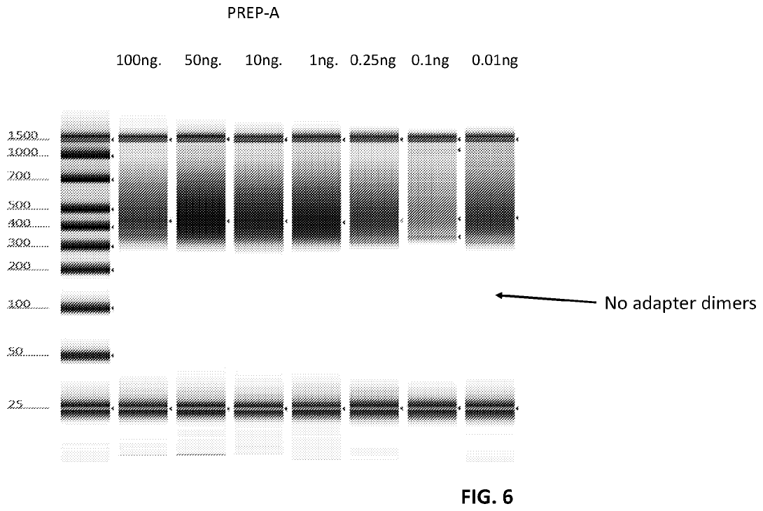

The schematic workflow is pictographically represented in FIG. 2 and FIG. 3 (steps 2-5). A sample reaction yielded PCR products as shown in FIGS. 5A-5B, using the protocol described above. The reactions were carried out a single vessel (one pot reaction), using a wide range of starting material, 10 pg-100 ng. No input quantitation was required. No adapter to template quantitation was required. The process is compatible for automation, with 8-addition only steps.

### Example 2: An Exemplary Method of Performing the Assay

Provided herein is a general protocol for performing the reaction. For the reactions, the reagents used are as follows. Hybrid A—N8 primer is used at 50 pmol per sample and is obtained from Integrated DNA Technologies). The stretch of Ns represent random primer portion, which is machine mixed and HPLC purified. App-B adapter (Integrated DNA Technologies) (50 pmol per sample) is used herein. Random portion of the sequence is machine mixed, HPLC purified. RNaseOUT (60 U per sample) (Thermo) cat no. 10777019, 5,000 U. Maxima II minus Reverse Transcriptase (100 U per sample) (Thermo) cat. no. EP0753, 4×10,000 U. Exonuclease I (20 U per sample) (Thermo) cat. no. EN0582, 20,000 U. Shrimp Alkaline Phosphatase (rSAP) (1 U per sample) (New England Biolabs) cat. no. M0371L, 2,500 U. T4 RNA Ligase 2 truncated (T4 RNL2 trunc.) (400 U per sample) (New England Biolabs) cat. no. M0242L, 10,000 U. Terminal deoxynucleotidyl Transferase (TdT) (40 U per sample) (New England Biolabs) cat. no. M0315L, 2,500 U. 2× Equinox mix (50 μL per sample) (Watchmaker Genomics) cat. no. 7K0014-384, 12 mL (25 μL per sample included in the reaction mixture). Cas9 Nuclease S. pyogenes (20 μM) (46 pmol per sample) cat. no. M0386M, 2,500 pmol. Proteinase K, (1 μL per sample) (Thermo) cat. no. EO0491, 1 mL or at. no. EO0491, 5×1 mL. PCR primers-standard Illumina primers full length barcoded (smaller p5 and p7 only primers). Purification of the ligated or amplified products is performed using Ampure XP SPRI beads.

An exemplary optimized protocol for the method steps are detailed below.

Fragmentation: The following reagents are combined in a tube or well (e.g. vessel)
2 μL—from above (100 ng human liver RNA)
1 μL hybrid A—N8 (50 μM)
2 μL 5× First Strand buffer (Maxima H minus)
5 μL

The reagents are incubated 1-4 minutes at 95° C., and placed on ice for 2 minutes. To 2 microliters of the sample, three microliters of the mixture prepared above are added. (Addition 1→3 μL to 2 μL sample)

The following reagents are mixed for Reverse Transcription Reaction (Addition 2)→
5 μL each
2.5 μL nuclease free water
1 μL 0.1 M DTT
0.5 μL RNaseOUT (40 U/μL)
0.5 μL 10 mM dNTPs
0.5 μL Maxima H minus (200 U/μL)
10 μL

After mixing, it is incubated at 25° C., 15 minutes, 50° C., 50 minutes, 75° C., 10 min. 1 μL Exonuclease I (Exo I) (20 U/μL) and 1 μL Shrimp Alkaline Phosphatase (rSAP) (1 U/μL) and then added and incubated at 37° C. for 30 minutes and 95° C. for 15 minutes. (Addition 3)→2 μL each.

Terminal Deoxynucleotidyl Transferase (TdT) Reaction. Addition 4)→8 μL e
12 μL from above
3.5 μL nuclease free water
2 μL 10×TdT buffer
0.5 μL 1 mM total rNTP (0.25 mM each)
2 μL Terminal Transferase enzyme (20 U/μL)
20 μL total volume

Following addition of 8 microliters of the TdT mixture to the previous vessel, it is incubated at 37° C. for 30 min

Adapter Ligation, T4 RNA Ligase 2 Trunc. Addition 5→20 μL Each
20 from above
4 μL 10×T4 RNL2 buffer
1 μL nuclease free water
1 μL App-B adapter (50 μM)
12 μL 50% PEG8000 (final reaction: 15% PEG8000)
2 μL T4 RNL2 trunc. (200 U/μL)
40 μL total vol
Incubated at 28° C. for 30 min.

### A. Ampure Bead Purification:

In order to perform the purification, 40 μL nuclease free water and 145 μL Ampure Beads XP (1.8×) are added to the tube and mixed, and then allowed to bind RNA/cDNA at room temp 10 minutes. Beads are washed 2 times with 150 μL 80% EtOH, air-dried for 8 minutes, eluted with 24 μL 10 mM Tris pH 8 (wait 5 minutes for elution) transfer 23 μL to fresh PCR tube.

### PCR1:

### Add the Following to the PCR Tube:

23 μL from above
25 μL 2× Equinox (Watchmaker Genomics)
1.25 μL PCR primer barcoded p7 (25 μM)
1.25 μL PCR primer barcoded p5 (25 μM)
50 μL total volume100 ng input: 6 cycles

### PCR Protocol (→PCR Protocol)

98° C., 2 minutes
→98° C., 20 seconds
→60° C., 30 seconds
→72° C., 45 seconds
72° C., 2 minutes
4° C., forever

In the next step, the PCR product is purified using magnetic beads: Add 30-40 μL beads (0.6×-0.8×) allow bind DNA 10 minutes, wash 2 times with 150 μL 80% EtOH (wait 30 sec on each wash), air-dry 6 minutes, and remove from magnet, add 13 μL 10 mM Tris pH 8 to elute PCR products, wait 5 minutes, and put back on magnet and transfer 12 μL to a fresh tube. (use 1 μL for Qubit).

### Ribodepletion

→RNP formation
2 μL 10× Cas9 buffer (Buffer™ r3.1)
3.9 μL Human Ribo Guide RNA
2.3 μL Cas9 Nuclease S. pyogenes (20 UM)
1 μL RNaseOUT (40 U/μL)
9.2 μL total volume

The above mixture is incubated at room temperature 10 minutes. The mixture is added to tube containing DNA˜11 μL volume from above pipetted up and down or flicked to mix. Incubated at 40° C. for 1 hour. 1 μL Proteinase K (20 mg/mL) is added and incubate at 56° C. for 10 min.

The reaction products are purified using magnetic beads: Add 30 μL NFW and 30 μL beads (0.6×), allow bind DNA 10 minutes, washed 2 times with 150 μL 80% EtOH (wait 30 sec on each wash), and removed from magnet, air-dried 6 minutes, 24 μL of 10 mM Tris pH 8 are added to elute PCR products, waited for 5 minutes and returned on magnet, then transferred 23 μL and to a fresh tube.

### PCR2:

The following reagents are added to the PCR tube (to make sure to use same barcodes as PCR1):
23 μL from above
25 μL 2× Equinox (Watchmaker Genomics)
1.25 μL PCR primer p5 (25 μM) use p5 behind the barcodes (bc)
1.25 μL PCR primer p7 (25 μM) use p7 behind bc
50 μL total volume
100 ng input: 7-8 cycles

### PCR Protocol→

### Hold 98° C., 2 Minutes

→98° C., 20 seconds
→60° C., 30 seconds
→72° C., 45 seconds
Hold 72° C., 2 minutes, then Hold 4° C., forever
Magnetic beads clean up is performed on PCR reaction:
50 μL beads (1×)

At this step, the DNA is allowed to bind for 10 minutes, washed 2 times with 200 μL 80% EtOH (wait 30 sec on each wash), air-dried for about 6 minutes, and removed from magnet add 26 μL 10 mM Tris pH 8 to elute PCR products. After waiting 5 minutes, it is put back on magnet then transfer 25 μL to a new tube. Qubit reading is taken on High sensitivity tape run or BioA (Load 2 ng per sample). Samples are pooled (perform second 06.×-0.8× cleanup if needed to get rid of any dimer product).

### Example 3: CDNA Library and Depletion Products-Qualitative Evaluation, Comparison 1

In this example, cDNA library prepared using the protocol described here is further subject to depletion of unwanted non-target sequences for downstream applications, following which the quality of the products are assessed to determine if the efficient and fast process for cDNA preparation described herein affects the quality of the final products.

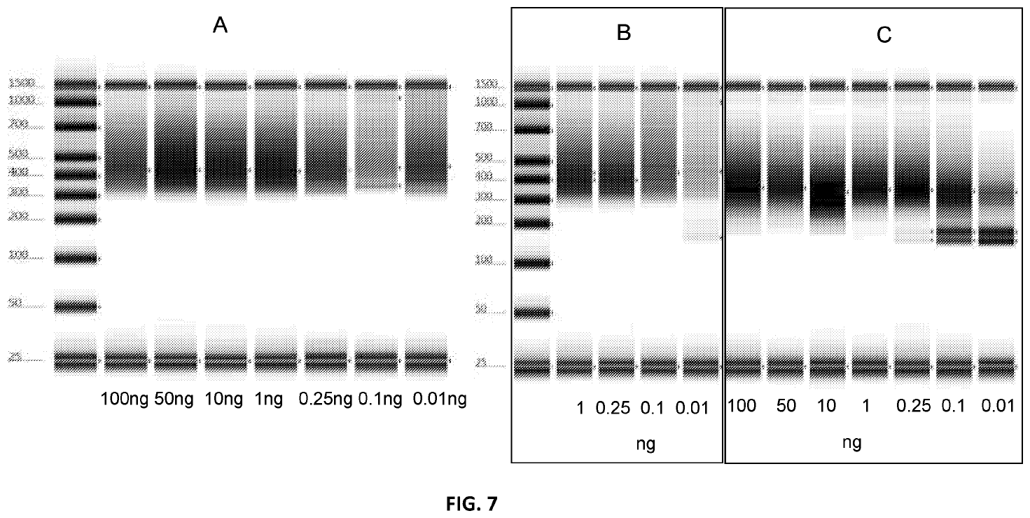

In a head to head comparison with a disclosed sample preparation protocol with other protocols (kits) from commercially available source, the instant protocol faired comparably, and in many aspects, significantly better. FIG. 7 shows the yields of each run with starting concentration as shown below the graphs. Preparations A (using instant protocol) was compared against third party commercially available protocols, B and C. Gel electrophoresis was performed as shown using the prepared and depleted libraries. The yields for A from 100 ng, 50 ng, 10 ng, Ing, 0.25 ng, 0.1 ng, 0.01 ng were respectively 73 ng, 88 ng, 80 ng, 85 ng, 58 ng, 45 ng, 195 ng. Average library size was about 520 bp, whereas that of C is about 400 bp. The respective yield in the compared case C were 83 ng, 115 ng, 170 ng, 232 ng, 67 ng, 37 ng, 24 ng at the same starting concentrations as for A and B. As evident in the electrophoresis data on the right side, primer dimerization is a problem for the lower concentration samples (last two), thereby compromising yield in C.

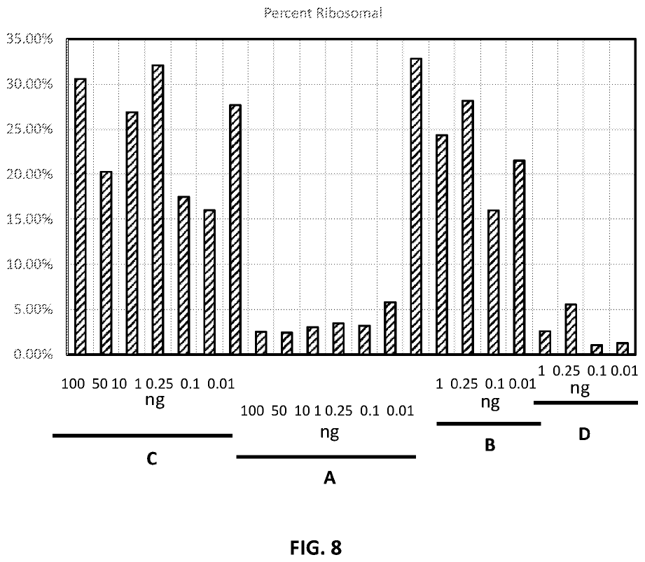

FIG. 8 shows percent ribosomal RNA remaining after in the sample after cDNA library preparation and depletion using the instant protocol A described herein; and comparing with other commercial protocols C and D. Here also, Protocol A (instant protocol) had much less ribosomal DNA left over after the depletion than B, C or D, thereby indicating that the cDNA preparation and depletion protocols of A worked better than the other competing ones.

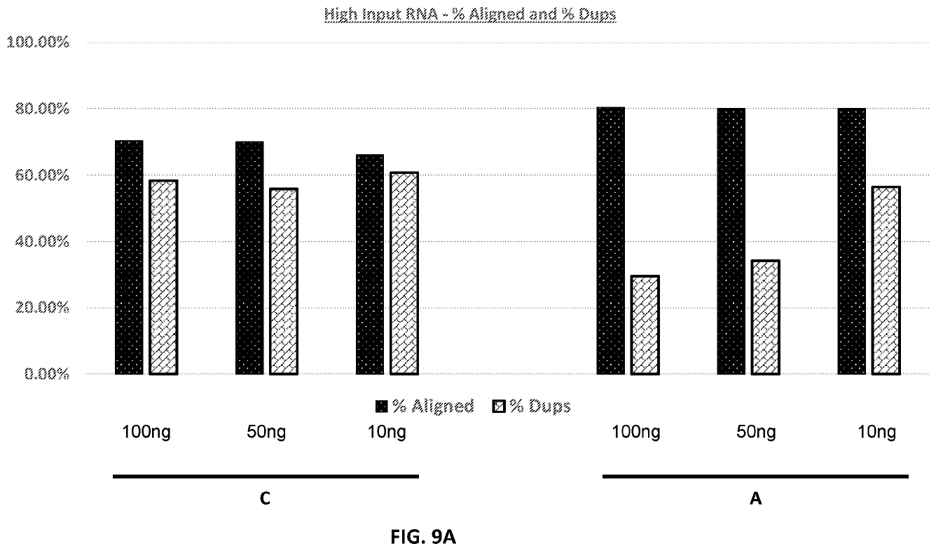

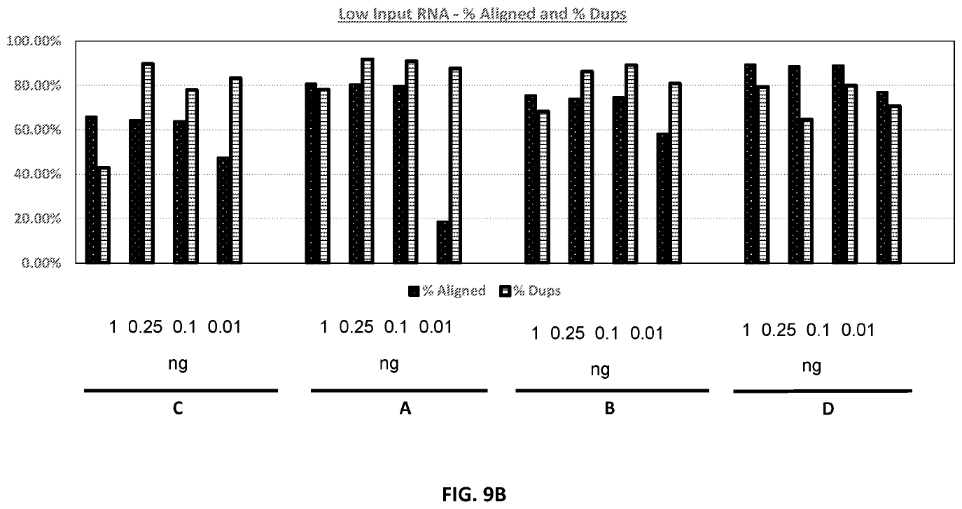

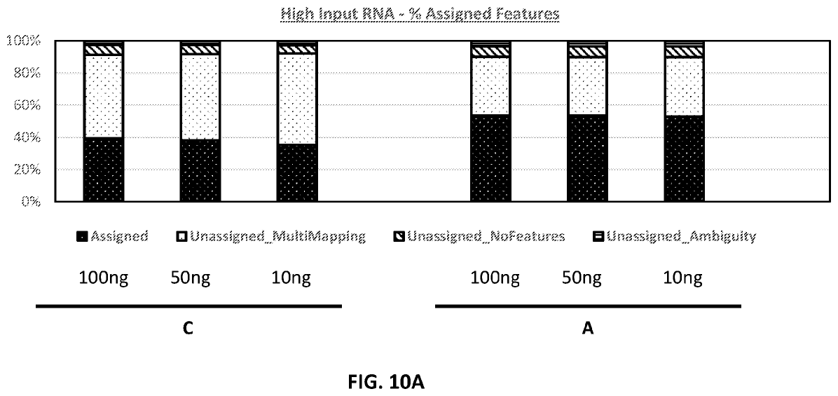

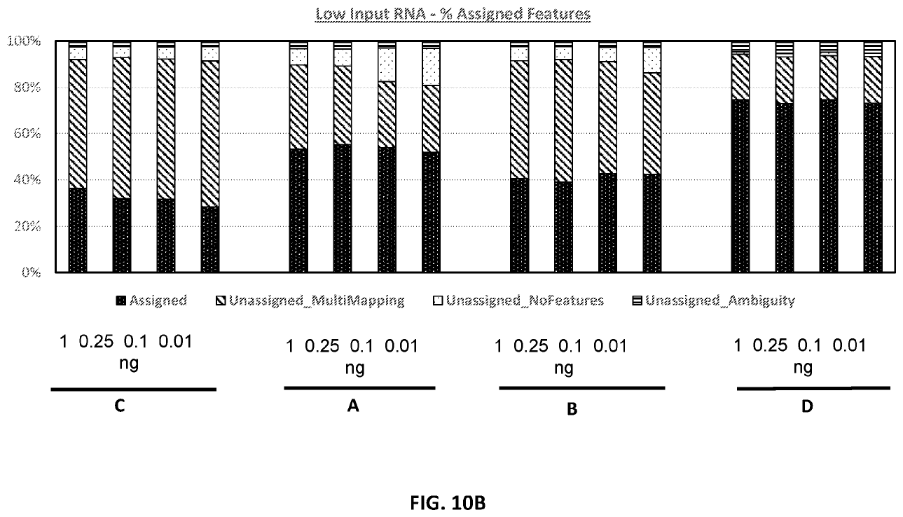

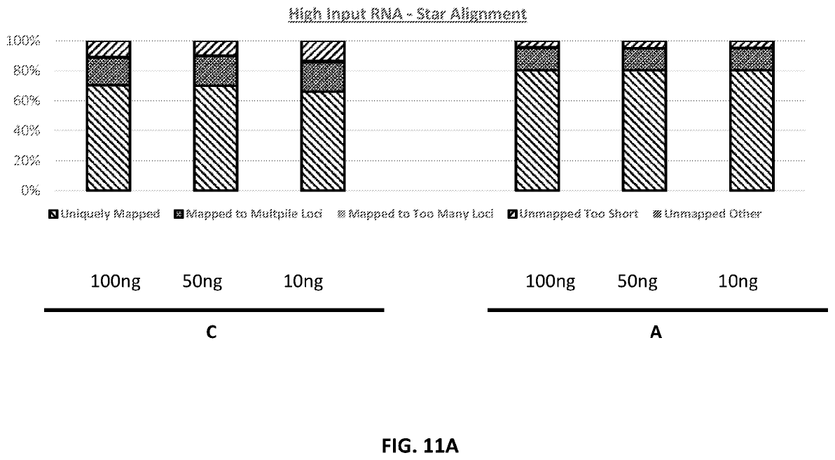

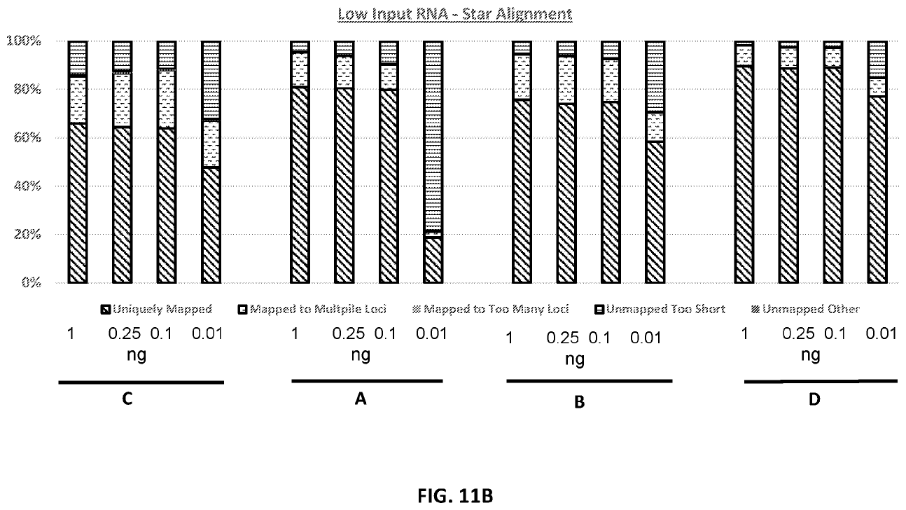

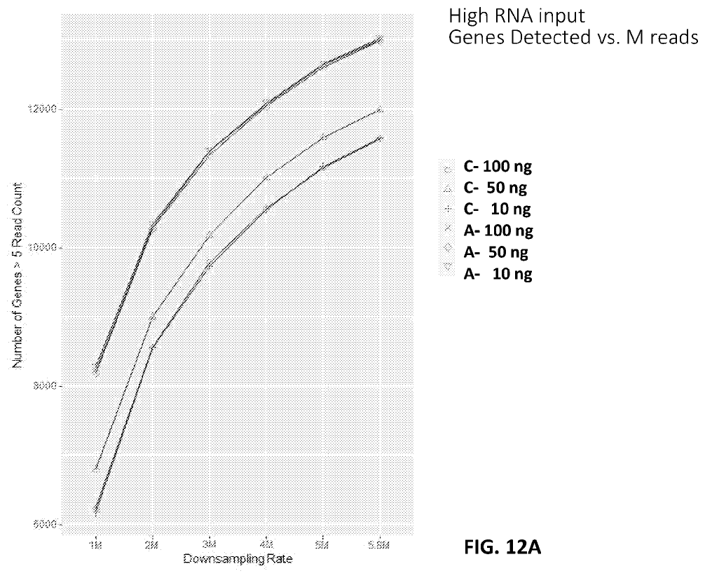

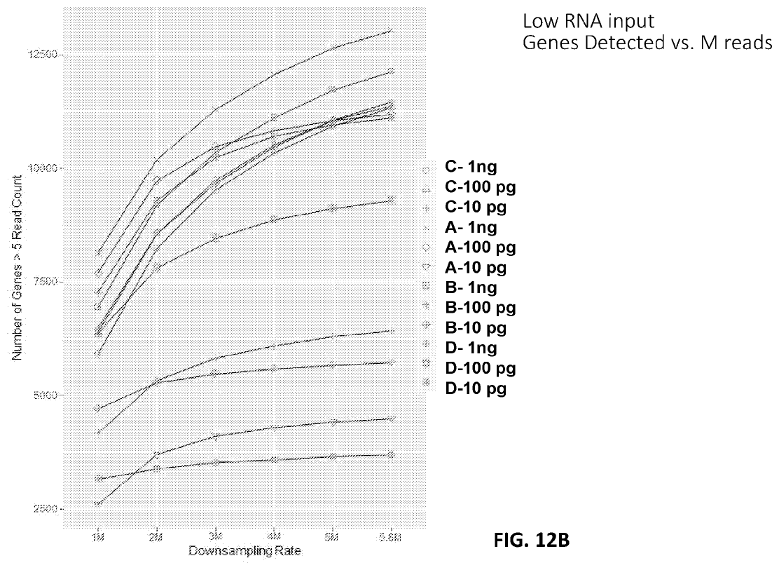

FIGS. 9A and 9B demonstrate the level of sample alignments and % duplicates after depletion reactions, comparing Protocols C with A. With high amount of input RNA, in particular, the instant protocol described herein yielded much higher sequence alignment percentages at the higher concentrations and much lower duplicate % with protocol A compared to protocol C (FIG. 9A). In case of the low input RNA, at least the % assigned was higher in the samples obtained using Protocol A compared to the others (FIG. 9B). Comparable trends were observed when the data were analyzed in details for percentages of sequences categorized as (a) assigned (b) unassigned, mapping to multiple regions, (c) unassigned, with no features, and (d) unassigned with ambiguity (FIGS. 10A and 10B). For high input RNA, as well as low input RNA samples, protocol A had an advantage over the other protocols in that there was a higher percentage of assigned and a lower percentage of unassigned multi-mapping sequences over the other compared outputs. Using star alignment program for sequence reads a very similar trend was obtained as FIGS. 10A and 10B, as demonstrated in FIGS. 11A and 11B respectively. Additionally, as shown in FIGS. 12A and 12B, the number of genes detected in the sequence reads confirm the findings shown in FIGS. 10A and 10B, and FIGS. 11A and 11B correspondingly for high input RNA and low input RNA respectively comparing the samples generated using the instant protocol A and other commercial protocols B-D.

### Example 4: CDNA Library and Depletion Products—Qualitative Evaluation—Comparison 2

In a different set of comparisons, the samples obtained using the instant protocol was compared head-to head with a different commercial protocol. In this study, a total of 15 samples were generated: 3 samples (N) and 12 samples (A) RNA input quantities for N were 100 ng, 50 ng, 10 ng; and for A were: 100 ng, 1 ng, 0.25 ng, 0.01 ng. respective RNP quantities for prep A: 0.6×→700 bp (fragment size); 0.7×→500 bp; 0.8×→400 bp. In an attempt to compare the performance of protocol N and utilizing the depletion protocol as discussed in the disclosure, with different RNA input quantities. For analysis, Protein counts and Guide counts are studied. As an overview, the results showed highest depletion at 0.6×RNP and 0.01 ng input quantity and better performance of instant protocol disclosed herein when compared to protocol N.

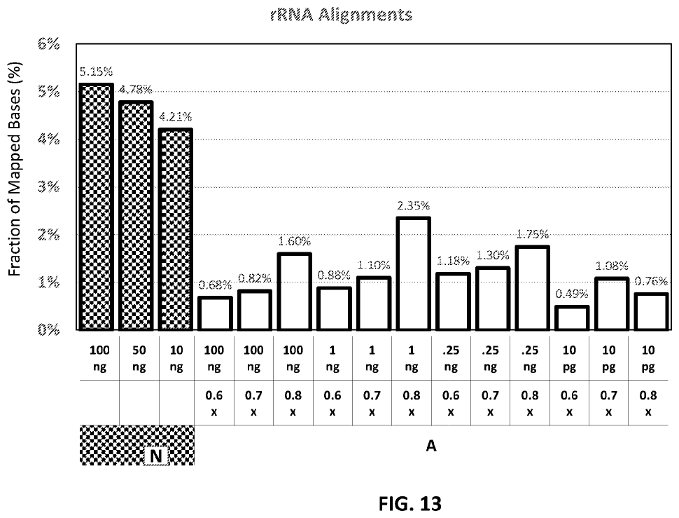

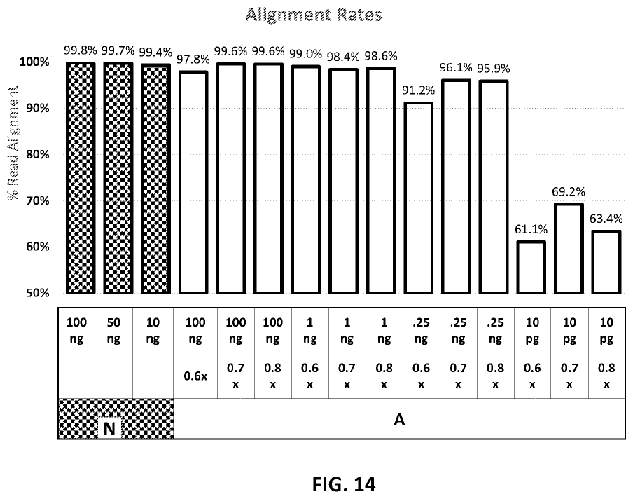

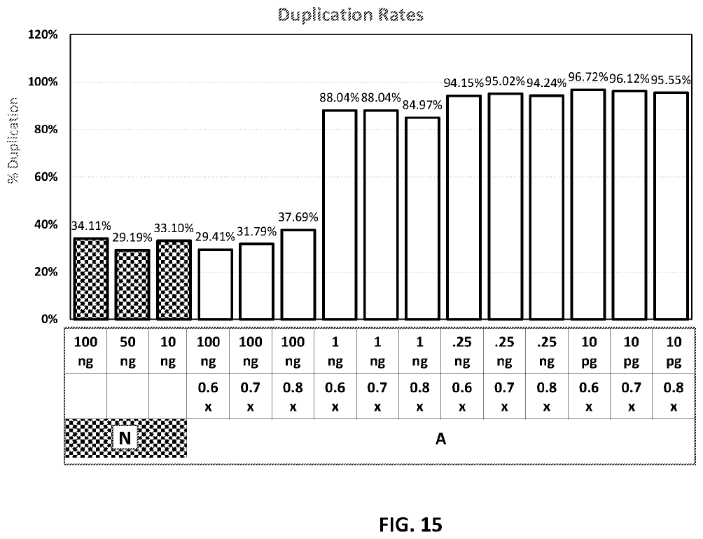

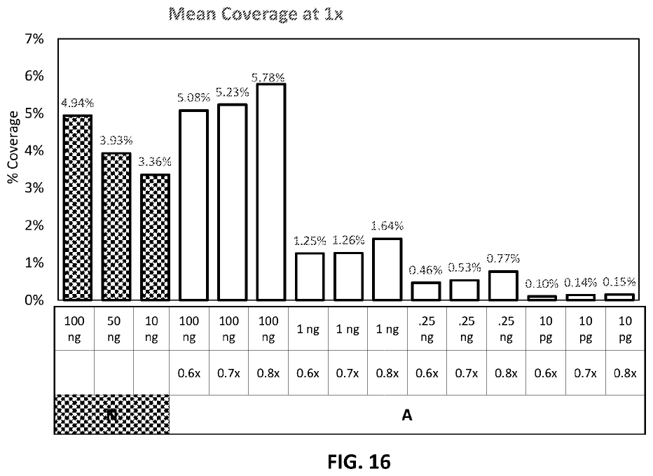

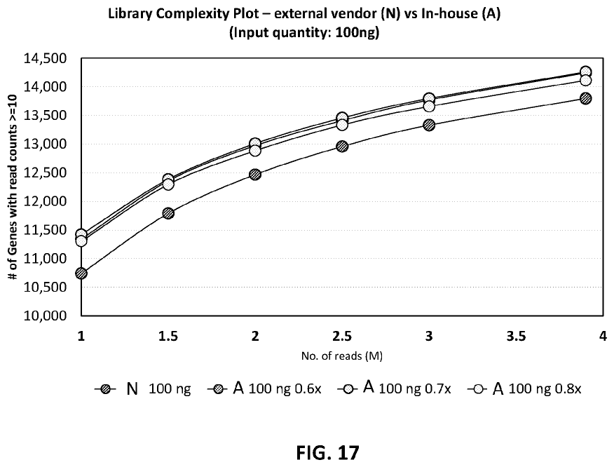

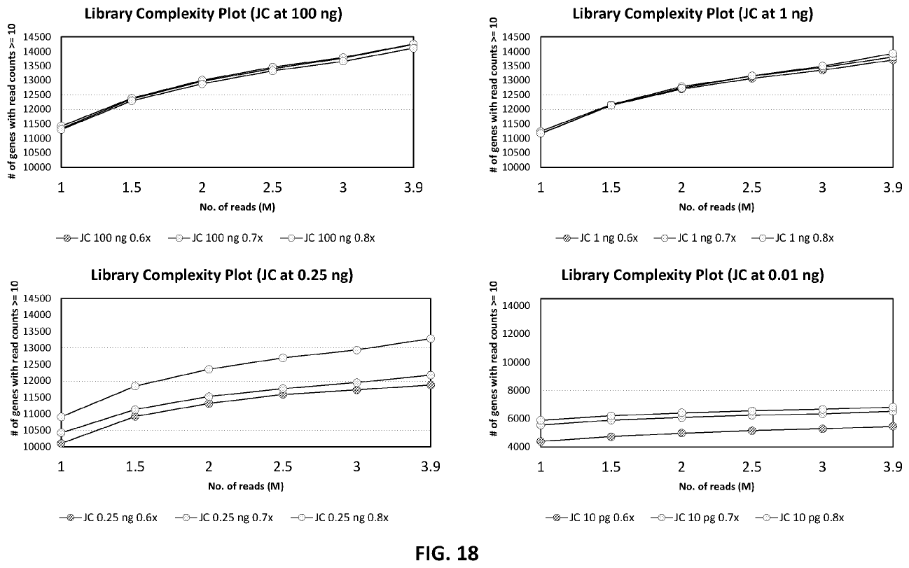

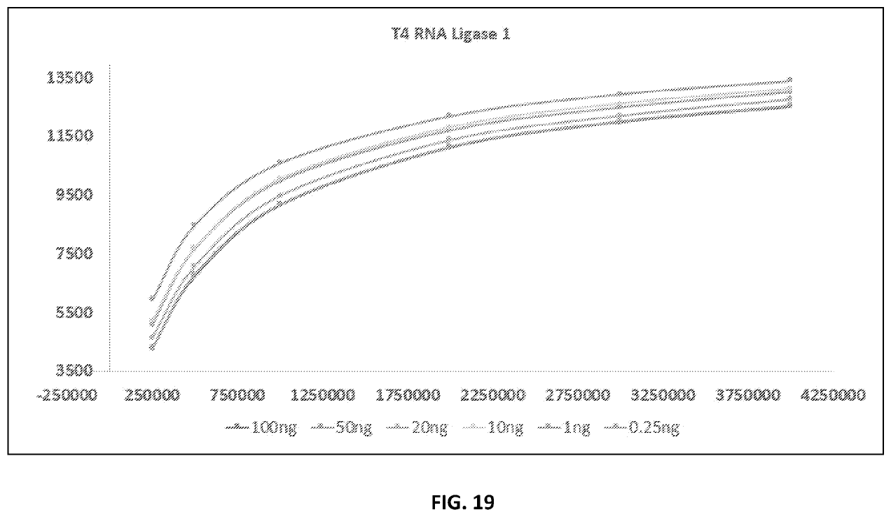

In a comparison of sequence alignment rates to ribosomal RNA, it was found that the instant protocol was significantly better since the depleted library sample from Protocol A carried much less of ribosomal sequences and much more of other sequences, which results in a higher the ratio of non-ribosomal (not depleted) to ribosomal (depleted) sequences in A compared to N (FIG. 13). High percentage reading of alignments in the two cases remained comparable (FIG. 14). In general, at the high input RNA concentrations, the % of duplicates were comparable between the two protocols (FIG. 15), and likewise, the mean coverage rates were slightly higher in the instant disclosed protocol (A) at high input RNA concentrations versus the commercial protocol N (FIG. 16). FIGS. 17 and 18 show the library complexity plot at different input RNA levels. In comparison, N samples had a slightly lower % of gene reads when sequenced, comparing gene sequences that were greater than 10 copy numbers in the sample than the A samples (FIG. 17). Within the preparations using protocol A, the quality of the reads decreased with the input RNA concentration and a small difference was observed between the 0.6× (700 bp fragment size); 0.7× (500 bp) and 0.8× (400 bp), as shown in FIG. 18. The guide counts plotted for 100 ng input for the 0.6× (700 bp fragment size); 0.7× (500 bp) and 0.8× (400 bp) respectively are shown in FIG. 18. FIG. 19 shows that T4 RNA ligase 1 could be successfully used in place of T4 RNA ligase 2 truncated. T4 RNA ligase 1 is used in combination with phosphorylated adapter, and is cost efficient over T4 RNA ligase 2 truncated and App-adapters.

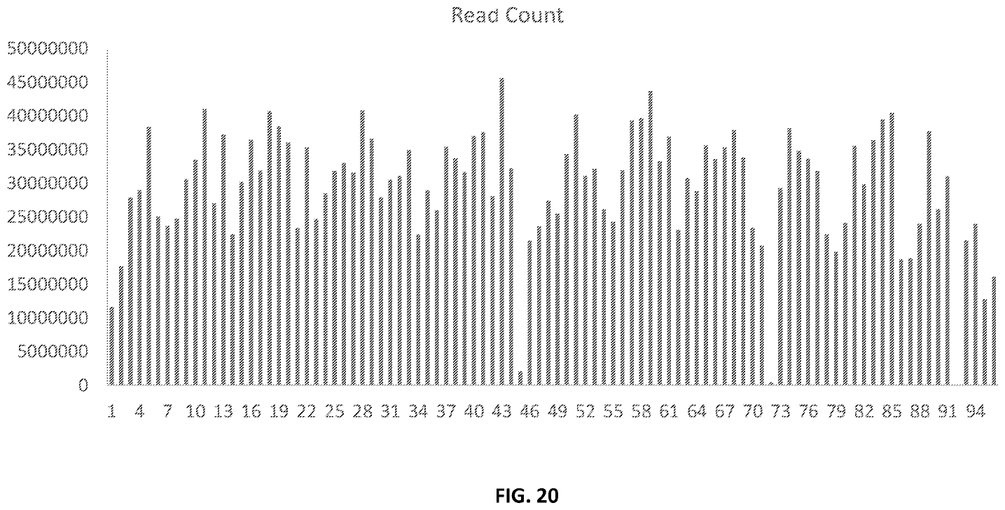

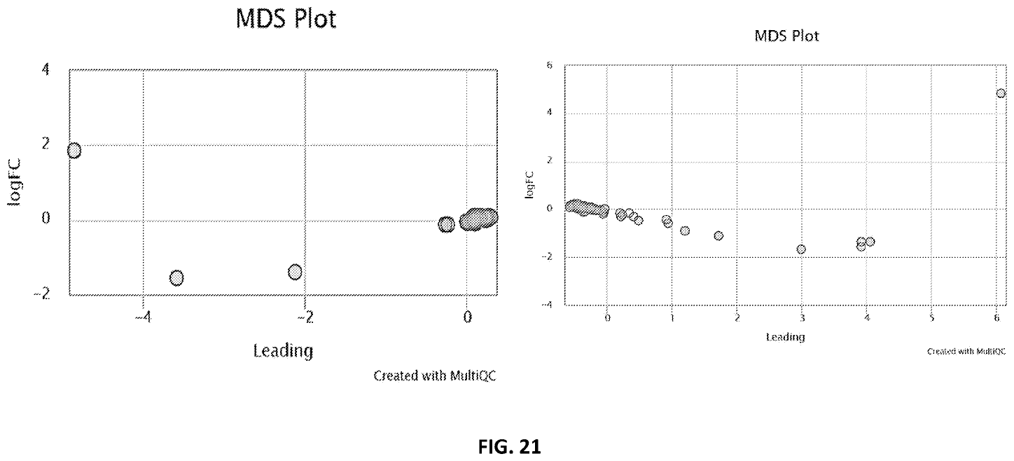

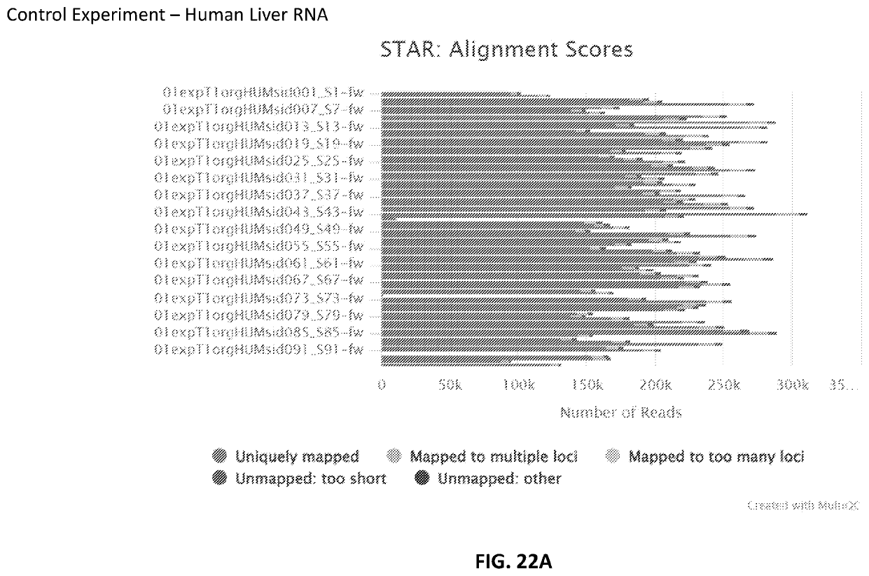

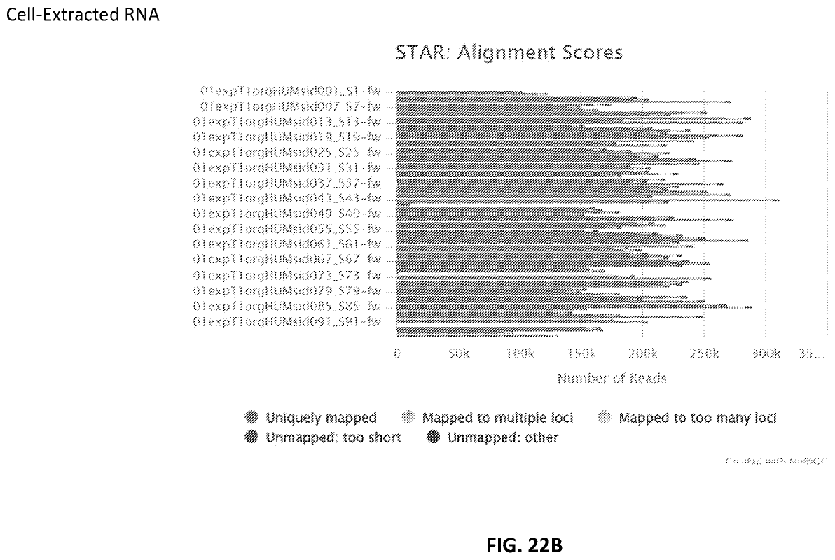

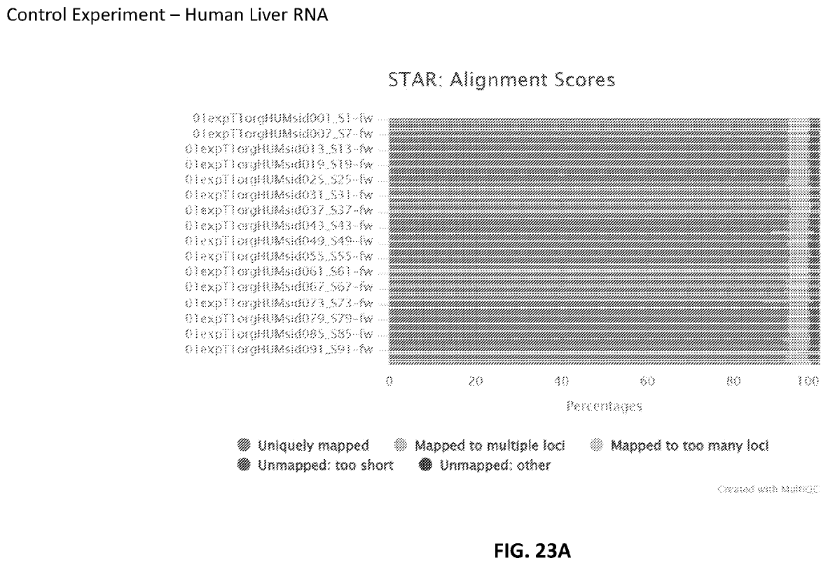

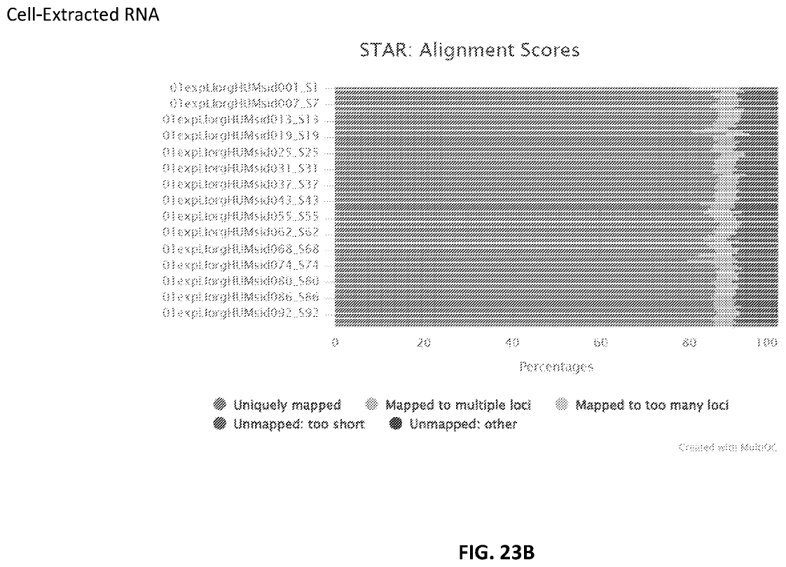

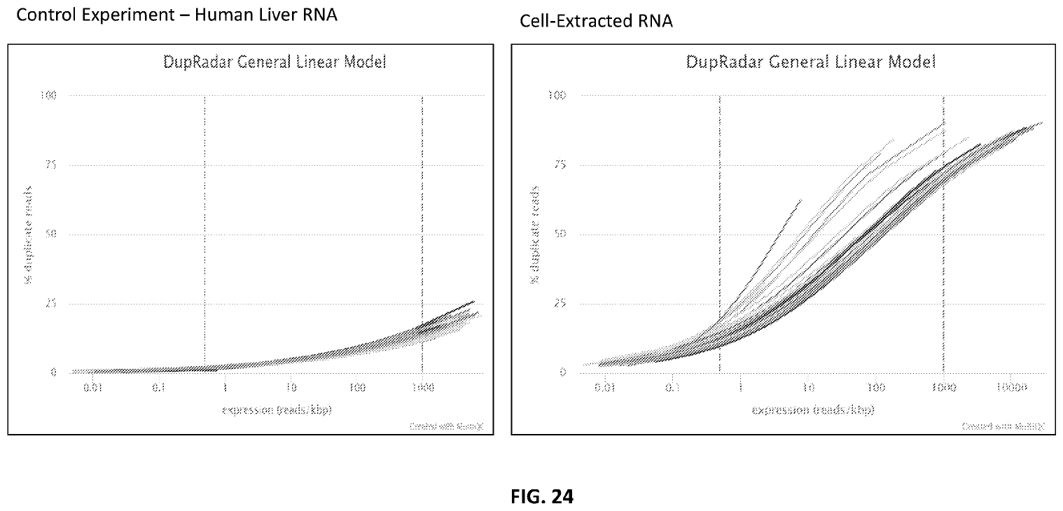

FIGS. 20-24 show RNA library preparation quality from RNA obtained from actual biological samples, e.g., liver RNA, and cell extracted RNA. The results demonstrate that the methods described herein lead to high quality sequencing reads in terms of count, percentage coverage of the genes identified and mapped.

### Example 5: An Exemplary Detailed Protocol for the PCT Amplified Product Generation

An exemplary reaction encompassing some of the steps described in the description is included in this section. However, this reaction is not to be considered as limiting disclosure in any sense.

### I. Reverse Transcription Reaction:

If working with low quantity samples (≤1 ng), a diluted solution of the random A—N8 primer [1 μL random A—N8 primer (50 μM) is used, diluted up to 250 μL with nuclease-free water] for any dilutions/transfers. The sequence of an exemplary random A—N8 primer: 5′TCCCTACACGACGCTCTTCCGATCTNNNNNNNN. This will act as a non-specific blocker to prevent loss of the sample to the walls of the pipette tips or tubes. To a 2 μL volume sample (0.1 ng-100 ng total RNA) the following are added: 0.5 μL random A—N8 primer (50 μM) 1 L 5× First Strand buffer; the 1.5 μL reaction is brought up to 3.5 μL final volume.

The above mixture is incubate at 94° C., 3 minutes followed by 4° C. or ice, 2 minutes. Then the following are added:

| | | |
|---|---|---|
| 0.5 | μL | 0.1M DTT |
| 0.25 | μL | nuclease-free water |
| 0.25 | μL | RNaseOUT (40 U/μL) |
| 0.25 | μL | 10 mM dNTPs |
| 0.25 | μL | Maxima H minus (200 U/μL) |
| 1.5 | μL | (5 μL final volume) |

Incubate at 25° C., 15 minutes, 50° C.; 50 minutes, 75° C. 10 minutes; final hold 4° C.

Add 0.5 μL Exonuclease I (20 U/μL) and 0.5 μL Shrimp Alkaline Phosphatase (rSAP) (1 U/μL).

Incubate at 37° C., 30 minutes; 95° C., 10 minutes; final hold 22° C. Do not stop here, go directly to next step.

### II. Terminal Deoxynucleotidyl Transferase (TdT) Reaction

To the 6 μL from above add the following:

| | | |
|---|---|---|
| 1.75 | μL | nuclease-free water |
| 1 | μL | 10X TdT buffer |
| 0.25 | μL | 1 mM total rNTP (0.25 mM each rATP, rCTP, rGTP, rUTP) |
| 1 | μL | Terminal deoxynucleotidyl Transferase (20 U/μL) |
| 4 | μL | (10 μL final volume) |

The mixture is incubated for 30 minute at 37° C.

III. cDNA Adapter Ligation Reaction

A 10×T4 RNL1 buffer, for example: 36 μL 10×T4 RNL buffer).

To the 10 μL from above the following are added and incubated at 28° C., for 30 minutes:

| | | |
|---|---|---|
| 2 | μL | 10X T4 RNA Ligase buffer (w/10 mM ATP; final 1 mM) |
| 0.5 | μL | nuclease-free water |
| 0.5 | μL | 5′-phosphorylated B adapter (50 μM) |
| 6 | μL | 50% PEG8000 (final reaction: 15% PEG8000) |
| 1 | μL | T4 RNA Ligase 1 (10 U/μL) |
| 10 | μL | (20 μL final volume) |

The reaction above is incubated at 28° C., 30 minutes. An exemplary 5′-phosphorylated B adapter sequence:/5Phos/NNNTGGAATTCTCGGGTGCCAAGGAA/3SpC3/

### Ampure Bead Purification:

To the 20 μL from above, 30 μL nuclease-free water and 90 μL AmpureXP beads (1.8×) is added and pipette mix up and down 10 times. The cDNA/RNA is allowed to bind to the AmpureXP beads at room temperature for 10 minutes. The tube is placed into a magnet and allow the solution to clear. Once it is clear, remove and discard the supernatant.
Without removing the tube from the magnet, the AmpureXP beads are washed two times with 200 μL of 80% ethanol solution. Wait 30 seconds during washes. The ethanol washings are removed and discarded. Care should be taken to remove all 80% ethanol solution following the second wash.
The tube is removed from the magnet, the cap of the tube is left open, and the AmpureXP beads are allowed to air-dry for 8 minutes.
To elute the cDNA/RNA, 24 μL of 10 mM Tris pH 8 is added and incubated for 5 minutes. The tube is returned back in the magnet and the solution allowed to clear. 23 μL of the cDNA/RNA is transferred to a fresh PCR tube.
1^{st} PCR: To the 23 μL sample from above the following are added:

| | | |
|---|---|---|
| 25 | μL | 2X Equinox buffer |
| 1.25 | μL | PCR primer p7 (barcoded)(25 μM)(full-length primers) |
| 1.25 | μL | PCR primer p5 (barcoded)(25 μM)(full-length primers) |
| ~50 | μL | total volume |

The number of cycles may vary depending on input (recommend: 5 cycles for 0.25 ng-100 ng and 10 cycles for <0.25 ng)

### PCR Protocol

98° C., 2 minutes; 98° C., 20 seconds; 55° C., 30 seconds; 68° C., 45 seconds; 72° C., 2 minutes; 4° C., final hold.
   Exemplary 1st PCR Primer p5-(Barcoded) has a Sequence:

5′AATGATACGGCGACCACCGAGATCTACACNNNNNNNNACACTCTTTCC CTACACGACGCTCTTCCGATCT (NNNNNNNN represents sequence of a barcode). Standard 501-508 8 base barcodes or 10 base 384 UDI barcodes can be used. Exemplary 1^{st} PCR primer p7-(barcoded) has a sequence 5′CAAGCAGAAGACGGCATACGAGATNNNNNNNNGTGACTGGAGTTCCTTGGCACC CGAGAATTCCA where (NNNNNNNN represents sequence of a barcode). A standard 701-712 8 base barcodes or 10 base 384 UDI barcodes can be used.

### Post-PCR1 Ampure Bead Purification:

40 μL AmpureXP beads (0.8×) are added to the PCR mix and pipetted up and down 10 times. The PCR product is allowed to bind to the AmpureXP beads at room temperature for 10 minutes. The tube is placed into a magnet and the solution is allowed to clear. Once it is clear, the supernatant is removed and discarded. Without removing the tube from the magnet, the AmpureXP beads are washed two times with 150 μL of 80% ethanol solution. Wait 30 seconds during washes. The ethanol washings are discarded. Care should be taken to remove all 80% ethanol solution following the second wash. The tube is removed from the magnet, and the cap of the tube is left open to allow the AmpureXP beads to air-dry for 5 minutes.

To elute the PCR product, 12 μL* of 10 mM Tris pH 8 is add and wait 5 minutes. The tube is returned back in the magnet and the solution is allowed to clear. At this time, 11 μL* of the PCR product is transferred to a fresh PCR tube. If sample input at step 1 is ≤1 ng, a second AmpureXP bead purification should be performed, the PCR product is eluted with 51 μL 10 mM Tris pH 8 and 50 μL is transferred to a fresh PCR tube and second AmpureXP bead clean procedure is performed: 40 μL AmpureXP beads (0.8×) is added and allowed to bind the DNA over 10 minutes, AmpureXP beads are wash 2 times with 150 μL 80% EtOH (wait 30 sec on each wash), air-dry for 5 minutes. After removing from magnet, 12 μL 10 mM Tris pH 8 is added to elute the PCR products, and after waiting 5 minutes, it is put back on magnet and 11 μL is transferred to a fresh PCR tube).

### Ribodepletion

In this step, guide RNAs are at incubated 70° C. for 2 minutes and then at 4° C. for 2 minutes. Then these are used directly in RNP formation.
→RNP formation
2 μL 10× Cas9 buffer
   3.9 μL Human Ribo Guide RNA
   2.3 μL Cas9
   1 μL RNaseOUT
   9.2 μL total volume
Incubate at room temp 10 minutes.
Add RNP mixture to tube containing PCR product, ˜11 μL volume from above, pipette up and down to mix.
Incubate 40° C. for 2 hours* for 1 ng-100 ng and 4 hours* for 0.01 ng-0.25 ng.
To quench reaction, add 1 μL Proteinase K and incubate at 56° C. for 10 minutes.

### Post-Ribodepletion Ampure Bead Purification:

30 μL nuclease-free water and 40 μL AmpureXP beads (0.8×) are added to the PCR mix and pipetted up and down 10 times. The PCR product is allowed to bind to the AmpureXP beads at room temperature for 10 minutes. Place the tube into a magnet and allow the solution to clear. Once it is clear, the supernatant is removed and discarded.

Without removing the tube from the magnet, the AmpureXP beads are washed two times with 150 μL of 80% ethanol solution with an interval of 30 seconds during washes.

The tube is removed from the magnet, and allowed to air-dry for 5 minutes. To elute the PCR product, 46 μL of 10 mM Tris pH 8 is added and we have to wait for 5 minutes.

The tube is placed back in the magnet and the solution is allowed to clear.

45 μL of the PCR product is transferred to a fresh PCR tube.

2nd PCR: The second PCT reaction is set forth as follows:
45 μL PCR product from above
50 μL 2× Equinox buffer
2.5 μL PCR primer p5 (25 μM) (p5 primer only-no barcode)
2.5 μL PCR primer p7 (25 μM) (p7 primer only-no barcode)
100 μL total volume
Number of cycles will vary depending on input (recommend: for 100-50 ng, 8-9 cycles, for 40-5 ng, 10-12 cycles, for 1-0.25 ng, 14-16 cycles, and for less than <0.25 ng, 11-14 cycles).

### PCR Protocol

98° C., 2 minutes; 98° C., 20 seconds; 55° C., 30 seconds; 68° C., 45 seconds: 72° C., 2 minutes; 4° C., final hold.

### Exemplary 2nd PCR Primer p5 Sequence is:

5′AATGATACGGCGACCACCGAGATCTACA

### Exemplary 2nd PCR Primer p5 Sequence is:

5′CAAGCAGAAGACGGCATACGAGA.

Post-PCR2 Ampure Bead purification: 100 μL AmpureXP beads (1×) are added to the PCR mix and pipetted up and down 10 times. The PCR product to allowed to bind to the AmpureXP beads at room temperature for 10 minutes. The tube is placed into a magnet and the solution allowed to clear. Once it is clear, the supernatant is discarded. Without removing the tube from the magnet, the AmpureXP beads are two times with 200 μL of 80% ethanol solution, and the AmpureXP beads are allowed to air-dry for 5 minutes. To elute the PCR product, 26 μL of 10 mM Tris pH 8 is added waited for 5 minutes and the tube is placed back in the magnet. Once the solution becomes clear transfer 25 μL of the PCR product to a fresh PCR tube. The product is analyzed in Qubit measurement and Run High Sensitivity Tapestation or BioAnalyzer. If too much dimer is present (with very low inputs) a 2nd 0.7× AmpureXP cleanup is performed.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A method of preparing a cDNA library, the method comprising:
(A) obtaining a plurality of RNA molecules from a sample;
(B) generating a first strand cDNA complementary to a RNA molecule from the plurality of RNA molecules using a random primer and a single stranded first adapter,
(C) incorporating a ribonucleotide tail using a mixture of ribonucleotide bases at the 3′-terminus of the first strand cDNA,
(D) adding a single stranded second adapter to the 3′-terminus of the ribonucleotide tail using an enzyme that requires a 3′-ribonucleotide terminus as an acceptor for ligation of the second adapter, thereby generating the first strand cDNA with single stranded adapters at both ends;
(E) amplifying the first strand cDNA, thereby preparing the cDNA library.

2. The method of claim 1, wherein the sample is a biological sample.

3. The method of claim 2, wherein the biological sample is a fresh biological sample, a frozen biological sample or a forensic sample.

4. The method of claim 1, wherein obtaining the plurality of RNA molecules comprises extracting total RNA from the sample.

5. The method of claim 1, wherein the single stranded first adapter is a universal adapter.

6. The method of claim 1, further comprising removing unused primers by an exonuclease and a phosphatase after generating the first strand cDNA.

7. The method of claim 1, wherein incorporating the ribonucleotide tail comprises incorporating less than 10 ribonucleotides at the 3′-terminus of the first strand cDNA.

8. The method of claim 7, wherein the ribonucleotide tail is incorporated using a terminal transferase (TdT).

9. The method of claim 1, wherein adding the single stranded second adapter to the 3′-terminus of the ribonucleotide tail comprises adding the single stranded second adapter to 3′-terminal ribonucleotide incorporated in step (C).

10. The method of claim 1, wherein amplifying comprises performing a polymerase chain reaction (PCR) using primers that anneal to the first adapter and the second adapter to generate an amplified double stranded cDNA.

11. The method of claim 1, wherein the cDNA library obtained from step (E) is first cDNA library that comprises at least one cDNA molecule comprising a target sequence and at least one cDNA molecule comprising unwanted non-target sequence, and wherein the method further comprises depleting a subset of the first amplified cDNA library comprising the unwanted non-target sequence from the first amplified cDNA library.

12. The method of claim 11, wherein depleting a subset of the first amplified cDNA library comprising the unwanted non-target sequences from the first amplified cDNA library is performed using a nucleic acid guided endonuclease.

13. The method of claim 12, wherein the nucleic acid guided endonuclease comprises a CAS endonuclease.

14. The method of claim 10, wherein the target nucleotide sequence comprises a pathogen sequence, and the depleting a subset of the first amplified cDNA library comprises depleting non-pathogen host genomic nucleic acid.

15. The method of claim 14, wherein the depleting a subset of the first amplified cDNA library comprises depleting contaminant human nucleic acid.

16. The method of claim 15, wherein contaminant human nucleic acid is ribosomal nucleic acid.

17. The method of claim 15, wherein contaminant human nucleic acid is a repeat nucleic acid sequence.

18. The method of claim 10, wherein the target nucleotide comprises a fetal nucleic acid and the depleting a subset of the first amplified cDNA library comprises depleting non-target contaminant maternal nucleic acid.

19. The method of claim 10, wherein the target nucleotide comprises a genomic polymorphism and wherein the depleting a subset of the first amplified cDNA library comprises depleting a wild type nucleic acid sequence.
